(19) United States
(12) Patent Application Publication
(10) Pub. No.: US 2022/0096484 A1
(43) Pub. Date: Mar. 31, 2022

(54) METHODS AND COMPOSITIONS FOR TREATING CANCER BY TARGETING FATP2 AND MYELOID DERIVED SUPPRESSOR CELLS

(71) Applicant: The Wistar Institute of Anatomy and Biology, Phitadelphia, PA (US)

(72) Inventors: Dmitry I. Gabrilovich, Villanova, PA (US); Filippo Veglia, Philadelphia, PA (US)

(21) Appl. No.: 17/429,718

(22) PCT Filed: Feb. 21, 2020
(86) PCT No.: PCT/US2020/019180

§ 371 Date: Aug. 10, 2021

Related U.S. Application Data
(60) Provisional application No. 62/830,506, filed on Apr. 7, 2019.
(60) Provisional application No. 62/808,787, filed on Feb. 21, 2019.

(51) Int. Cl. A61K 31/519; A61K 31/433; A61K 39/395; C07K 16/28; A61P 35/00

(52) U.S. Cl. CPC A61K 31/519; A61K 31/433; A61P 35/00; C07K 16/2818; C07K 16/2866; A61K 39/3955

(57) ABSTRACT

A method of treating cancer in a subject in need thereof is provided. The method includes administering an agent that inhibits, decreases, deletes or downregulates FATP2. In one embodiment, the method includes administering a checkpoint inhibitor. Also provided are methods of decreasing MDSCs and MDSC immune-suppressive activity in a subject in need thereof.

### GOVERNMENT LICENSE RIGHTS

This invention was made with government support under grant numbers CA R01CA165065 and AI110485 awarded by the National Institutes of Health. The government has certain rights in the invention.

### BACKGROUND OF THE INVENTION

Myeloid-derived suppressor cells (MDSC) represent a heterogeneous population of immature myeloid cells. These cells accumulate to a great extent in cancer patients and play a major role in regulating immune responses in cancer. MDSC suppress T cell activation and proliferation as well as function of natural killer (NK) cells. Ample evidence links these cells with tumor progression and outcome of the disease in cancer patients. The accumulation of relatively immature and pathologically activated myeloid-derived suppressor cells (MDSC) with potent immunosuppressive activity is common in tumors. MDSC have the ability to support tumor progression by promoting tumor cell survival, angiogenesis, invasion of healthy tissue by tumor cells, and metastases. There is now ample evidence of the association of accumulation of immune suppressive MDSC with negative clinical outcomes in various cancers. MDSC have been implicated in resistance to anticancer therapies, including kinase inhibitors, chemotherapy, and immune therapy.

### SUMMARY OF THE INVENTION

Provided herein is a method of decreasing MDSCs in a subject in need thereof. The method includes inhibiting, decreasing, deleting, or downregulating FATP2.

In another aspect, a method of decreasing the immune-suppressive activity of PMN-MDSCs in a subject in need thereof is provided. The method includes inhibiting, decreasing, deleting, or downregulating FATP2.

In yet another aspect, a method of treating cancer in a subject in need thereof is provided. The method includes inhibiting, decreasing, deleting, or downregulating FATP2.

In another aspect, method of decreasing arachidonic acid or arachidonic acid-containing phospholipids in a subject in need thereof comprising inhibiting, decreasing, deleting, or downregulating FATP2.

In yet another aspect, a method of decreasing PGE2 synthesis in a subject in need thereof is provided. The method includes inhibiting, decreasing, deleting, or downregulating FATP2.

In one embodiment, the method includes administering a FATP2 inhibitor to a subject. In another embodiment, the inhibitor is lipofermata or grassofermata. In another embodiment, the method further includes administering a checkpoint inhibitor to the subject. In another embodiment, the subject has cancer.

Other aspects and advantages of these compositions and methods are described further in the following detailed description of the preferred embodiments thereof.

### BRIEF DESCRIPTION OF THE DRAWINGS

FIG. 1A-FIG. 1H demonstrate the effect of FATP2 deletion on tumor growth and PMN-MDSC function. FIG. 1A, Slc27a2 expression in control (Ctrl) PMNs and PMN-MDSCs from the spleen or tumors of tumor-bearing mice. FIG. 1B, Slc27a2 expression in M-MDSCs and PMN-MDSCs from the spleen of tumor-bearing mice. FIG. 1C, Slc27a2 expression in indicated cells in EL4 tumor-bearing mice. TAM, tumor-associated macrophages. In FIG. 1A-FIG. 1C, results of individual mice are shown (n=4-5). FIG. 1D, LLC or EL4 tumor growth in wild-type (WT) or FATP2-knockout (Slc27a2^{-/-}) C57BL/6 mice (n=4-5). Representative of two experiments. FIG. 1E, EL4 tumor growth in mice reconstituted with bone marrow cells from wild-type or Slc27a2 mice (n=4-5). Representative of two experiments. FIG. 1F, LLC or EL4 tumors in wild-type and Slc27a2^{-/-} mice depleted of CD8 T cells using an anti-CD8 (aCD8) antibody. Representative of two experiments (n=4-5). FIG. 1G, LLC tumors in Slc27a2^{fl/fl} mice crossed with S100a8-cre mice, to target the FATP2 depletion to PMNs (n=4). FIG. 1H, Suppression of T cell proliferation in PMN-MDSCs isolated from wild-type or FATP2-knockout (Slc27a2^{-/-}) tumor-bearing mice. Proliferation was determined by incorporation of [³H]thymidine. CPM, counts per min. Four experiments were performed with similar results. Dashed line shows T cell proliferation without MDSCs. *P<0.05, **P<0.01, ***P<0.001, ****P<0.0001 (between control and test samples), unpaired two-sided Student's t-test (FIG. 1A-FIG. 1C) or two-way analysis of variance (ANOVA) (FIG. 1D-FIG. 1D).

FIGS. 2A-FIG. 2L demonstrate the mechanism of FATP2 mediated suppression by PMN-MDSC. FIG. 2A, Triglycerides (TG) in PMN-MDSCs from spleens of LLC wild-type (n=7) and FATP2-knockout (Slc27a2^{-/-}) tumor-bearing mice (n=6). TG(AA), triglycerides containing arachidonic acid; TG(56:8), triacylglycerol. FIG. 2B, Fatty acids in PMNMDSCs from the spleen of wild-type (n=12) and Slc27a2^{-/-} (n=11) tumor-bearing mice. PUFA, polyunsaturated fatty acid. FIG. 2C, Phospholipid species containing arachidonic acid (AA) residues in PMN-MDSCs from spleen of wild-type (n=12) and Slc27a2^{-/-} (n=10) tumor-bearing mice. PC, phosphatidylcholine; PE, phosphatidylethanolamine. FIG. 2D, AA-d₁₁ and PGE₂-d₁₁ in PMN-MDSCs from spleen of wild-type (n=5) and Slc27a2^{-/-} (n=5) tumor-bearing mice. FIG. 2E, AA-d₁₁-labelled phosphatidylethanolamine and phosphatidylcholine in PMN-MDSCs from wild-type (n=5) and Slc27a2^{-/-} (n=4) tumor-bearing mice. FIG. 2F, PGE₂ (determined by LS-MS) in PMN-MDSCs from wild-type (n=12) and Slc27a2^{-/-} mice (n=11). FIG. 2G, Relative expression of Slc27a2 (determined by qRT-PCR) in PMNs generated from HPCs transduced with lentivirus expressing Slc27a2-gfp (GFP⁺) or control lentivirus (GFP⁻) (n=4). FIG. 2H, PGE₂ release from cells described in FIG. 2G (n=4). Fold change compared with control GFP⁻ cells after transduction. FIG. 2I, Suppression of T cell proliferation (in triplicates) of PMNs differentiated from HPCs in the presence of arachidonic acid. Representative of three experiments. Dashed line shows T cell proliferation without MDSCs. FIG. 2J, PGE₂ production by PMNs differentiated from HPCs in the presence of arachidonic acid (n=5). Fold change compared with control. FIG. 2K, PGE₂ production by PMNs differentiated from Ptgs2 HPCs in the presence of arachidonic acid (n=4). Fold change compared with control. FIG. 2L, Suppression of T cell proliferation (in triplicates) of PMNs differentiated from Ptgs2^{-/-} HPCs in the presence of arachidonic acid. Representative of two independent experiments. Dashed line shows T cell proliferation without MDSCs. Data are mean±s.d. *P<0.05, **P<0.01, ***P<0.001, ****P<0.0001 (between control and test samples), unpaired two-sided Student's t-test. In FIG. 2A-FIG. 2F concentration pmol/mg of protein is shown.

FIG. 3A-FIG. 3K show the regulation of FATP2 in PMN-MDSC. FIG. 3A, FATP2 protein in PMNs treated with GM-CSF. β-actin was used as a loading control. Representative of three experiments. FIG. 3B, ChIP assay using an anti-STAT5 antibody in PMNs isolated from the bone marrow of tumor-free mice treated with GM-CSF. Triplicate measurements, representative of two experiments. FIG. 3C, FATP2 protein in PMNs from STAT5-knockout (KO; Stat5^{fl/fl}×S100a8-cre) mice treated with GMCSF. Representative of three experiments. FIG. 3D, Lipid accumulation (determined by BODIPY staining) in PMN-MDSCs isolated from the blood of healthy individuals (n=9) or patients with head and neck cancer (n=11), non-small cell lung cancer (n=6), or breast cancer (n=5). MFI, mean fluorescent intensity. FIG. 3E, Lipid accumulation (BODIPY staining) in PMN-MDSCs isolated from blood and tumor tissue of patients with non-small cell lung cancer (n=4). FIG. 3F, Expression of SLC27A2 (determined by RT-qPCR) in PMN-MDSCs isolated from the blood of patients with cancer or in PMNs of healthy donors. Fold change compared with control PMNs (n=6). FIG. 3G, FATP2 protein in PMN-MDSCs isolated from the blood of patients with cancer or in PMNs of healthy individuals. Representative of three experiments. FIG. 3H, SLC27A2 (RT-qPCR) in LOX1⁺ and LOX1⁻ PMNs from the blood of patients with cancer. Fold change compared with LOX1⁻ PMNs (n=8). FIG. 3I, LS-MS lipidomics analysis of triglycerides in PMNs from healthy donors and PMN-MDSCs from patients with cancer. n=4. FIG. 3J, LS-MS lipidomics analysis of free arachidonic acid, linoleic acid (LA), and docosahexaenoic (DHA) in PMNs from healthy donors and in PMN-MDSCs from patients with cancer (n=4). FIG. 3K, LS-MS lipidomics analysis of PGE₂ in PMNs from healthy donors and in PMN-MDSCs from patients with cancer (n=4). Data are mean±s.d. *P<0.05, **P<0.01, ***P<0.001, ****P<0.0001, two-way ANOVA (FIG. 3D) or unpaired two-sided Student's t-test (FIG. 3E, FIG. 3F, FIG. 3H-FIG. 3K). In FIG. 3I, FIG. 3J, FIG. 3K concentration pmol/mg of protein is shown.

FIG. 4A-FIG. 4E demonstrate the therapeutic effect of targeting FATP2. FIG. 4A-FIG. 4E, Treatments with lipofermata (2 mg kg⁻¹ twice per day subcutaneously) started 8-10 days after tumor injection. Anti-CTLA4 antibody (200 μg per mouse, intraperitoneally) was administrated at days 7 and 11 after tumor injection, and anti-CSF1R antibody (300 m per mouse, intraperitoneally) was administered every other day, starting the day after tumor injection. FIG. 4A, Growth of indicated tumors in C57BL/6 mice treated with lipofermata. Representative of two independent experiments (n=4-5 mice per group). FIG. 4B, Growth of LLC tumors in NOD-SCID mice treated with lipofermata (n=5). FIG. 4C, Growth of LLC tumors in mice depleted of CD8 T cells and treated with lipofermata (n=5). FIG. 4D, Growth of LLC tumors in mice treated with anti-CTLA4 antibody and lipofermata (n=5). FIG. 4E, Growth of LLC tumors in mice treated with CSF1R inhibitor and lipofermata (n=5). Data are mean±se.m. *P<0.05, **P<0.01, ***P<0.001, ****P<0.0001 (differences from untreated cells and between treated groups), two-way ANOVA with corrections for multiple comparison. NS, not significant.

FIG. 5A-FIG. 5H demonstrate lipid accumulation and expression of lipid transporters in MDSC. FIG. 5A, Lipid accumulation (BODIPY staining) in PMN-MDSCs isolated from the spleen and tumors of indicated tumor models (n=4-8 mice per group). Each circle represents an individual mouse. Inset shows confocal image representative of two independent experiments. FIG. 5B, Lipid accumulation (BODIPY staining) in PMNs generated from bone marrow HPCs treated with GM-CSF and tumor explant supernatant (n=3-5). FIG. 5C, LC-MS analysis of triglycerides in PMNs from control mice and PMN-MDSCs from EL4 tumor-bearing mice (n=4). FIG. 5D, Lipid accumulation (BODIPY staining) in M-MDSCs isolated from spleen and tumor of indicated tumor models (n=10). Each circle represents an individual mouse. FIG. 5E, Lipid accumulation (BODIPY staining) in dendritic cells (DCs) and MDSCs generated from CD204-deficient (Msr1^{-/-}) HPCs in the presence of tumor explant supernatant (n=3). FIG. 5F, Lipid accumulation in PMN-MDSCs from the spleen of wild-type and CD204-knockout (Msr1^{-/-}) tumor-bearing mice (n=3). FIG. 5G, Suppression of T cell proliferation of PMN-MDSCs from the spleen of wild-type and CD204-knockout tumor-bearing mice. Representative of four experiments, each performed in triplicate. FIG. 5H, Expression of Msr1, Fabp1, Fabp3, Fabp4, Fabp5, Slc27a1, Slc27a4, Slc27a5 and Cd36 in control PMNs and PMN-MDSCs isolated from the spleen and tumors of EL4 tumor-bearing mice (n=4-5). Data are mean±s.d. *P<0.05, **P<0.01, ***P<0.001, ****P<0.0001, unpaired two-sided Student's t-test.

FIG. 6A-FIG. 6K demonstrate expression of gene involved in lipogenesis in PMN-MDSC. FIG. 6A, FATP2 protein in control PMNs and PMN-MDSCs from spleen of tumor-bearing mice. Representative of two experiments. FIG. 6B, FATP2 protein in PMNs generated in vitro from bone marrow HPCs. Representative of two experiments. FIG. 6C, F244 tumor growth in wild-type and Slc27a2^{-/-} (FATP2 knockout) mice on a SV129 background (n=4). FIG. 6D, Verification of correct targeting of Slc27a2 by RT-qPCR and FATP2 by western blot analysis in PMN-MDSCs isolated from the spleen of Slc27a2^{fl/fl}×S100a8-cre⁻ and Slc27a2^{fl/fl}×S100a8-cre⁺ tumor-bearing mice. FIG. 6E, IFNγ production by CD8 T cells and CD4 and CD8 T cell proliferation (n=3) in wild-type and Slc27a2^{-/-} mice. FIG. 6F, Suppressive activity of M-MDSCs isolated from wild-type or Slc27a2^{-/-} tumor-bearing mice. Dashed line shows T cell proliferation without MDSCs. Four experiments were performed with similar results. FIG. 6G, Suppression of T cell proliferation of tumor-associated macrophages (TAM) from wild-type or Slc27a2^{-/-} tumor-bearing mice. Dashed line shows T cell proliferation without macrophages. Representative of three independent experiments. FIG. 6H, Growth of EL4 tumors in wild-type and Slc27a2^{-/-} mice (n=4). Representative of two experiments. FIG. 6I, Suppression of T cell proliferation of PMN-MDSCs isolated from the spleen or tumors of wild-type or Slc27a2^{-/-} mice. Representative of two independent experiments, performed in triplicate. Dotted line shows T cell proliferation without PMN-MDSCs. FIG. 6J, Growth of LLC tumors in wild-type and Cd36^{-/-} mice, depleted of CD8⁺ T cells (n=3). FIG. 6K, Lipid accumulation (BODIPY staining) in PMN-MDSCs and M-MDSCs isolated from the spleen and tumors of Cd36^{-/-} mice (n=3). Data are mean±s.d. ****P<0.0001, unpaired two-sided Student's t-test.

FIG. 7A-FIG. 7C show the effect of FATP2 KO on mRNA gene expression. FIG. 7A, Expression heatmap for genes affected by FATP2 depletion by at least fivefold. FIG. 7B, Number of significantly affected genes (FDR<5%) for different fold change thresholds. FIG. 7C, List of upstream regulators whose targets were found by ingenuity pathway analysis as significantly enriched among genes affected by FATP2 knockdown. n, number of affected targets; p, enrichment P value; Z, activation z scores calculated by IPA represent predicted regulator state based on the known effect on target and direction of mRNA change. Negative activation z scores predict inhibition, and positive z scores denote activation of the regulator in the FATP2-knockout mice.

FIG. 8A-FIG. 8F show the results of LC-MS analysis of lipids from WT and Slc27a2^{-/-} PMN-MDSCs. FIG. 8A, Triglycerides (TG) in PMN-MDSCs from spleens of EL4 wild-type (n=7) and FATP2-knockout (Slc27a2^{-/-}) tumor-bearing mice (n=6). Triglyceride species containing linoleic acid (18:2), docosapentaenoic acid (22:5), and docosahexaenoic acid (22:6) (n=7). FIG. 8B, Cholesterol esters in PMN-MDSCs from spleens of LLC wild-type (n=7) and FATP2-knockout (Slc27a2^{-/-}) tumor-bearing mice (n=6). Total cholesterol ester (CE) and arachidonoyl-containing (20:4) cholesterol ester. FIG. 8C, Fatty acids in PMN-MDSCs from the spleen of wild-type (n=12) and Slc27a2^{-/-} (n=11) tumor-bearing mice. Linoleic acid (18:2), docosapentaenoic acid (22:5), and docosahexaenoic acid (22:6) fatty acids. FIG. 8D, Distribution of major phospholipids in PMN-MDSCs from Slc27a2^{-/-} (FATP2 KO) (n=10) and wildtype (n=12) mice. PC, phosphatidylcholine; PE, phosphatidylethanolamine; PI, phosphatidylinositol; PS, phosphatidylserine. Subscript ‘AA’ denotes arachidonoyl-containing. FIG. 8E, Content of phospholipids containing arachidonic acid in PE, PC, PI and PS (n=12 for wild-type and n=10 for FAT2 KO mice). FIG. 8F, Content of AA-d₁₁-labelled phospholipids (PI, phosphatidylglycerol (PG), phosphatidic acid (PA) and PS), n=5. Data are mean±s.d.; each circle indicates an individual mouse. *P<0.05, **P<0.01, unpaired two-sided Student's t-test.

FIG. 9A-FIG. 9D show the results of metabolomic analysis and expression of FAO related genes in PMN-MDSC. FIG. 9A, Oxygen consumption rate (OCR) and basal OCR of wild-type and Slc27a2^{-/-} (FATP2 KO) PMN-MDSCs. Representative of two experiments (n=3-4). Cumulative results shown, and each circle indicates an individual mouse (n=7). No statistical differences (P>0.05) were determined by unpaired two-sided Student's t-test. FIG. 9B, Extracellular acidification rate (ECAR) and basal ECAR of wild-type and FATP2-knockout PMN-MDSCs. Representative of two independent experiments (n=3-4). Cumulative results shown, and each circle indicates an individual mouse (n=7). *P<0.05, unpaired two-sided Student's t-test. FIG. 9C, ¹³C-labelling of intermediates and associated amino acids of the tricarboxylic acid cycle. Ex vivo MDSCs were cultured in physiological-like medium supplemented with BSA-conjugated ¹³C₁₆-palmitate and GM-CSF for 18 h. Metabolites were then extracted and analysed by high-resolution LC-MS. ¹³C isotopologues (M+x) for each metabolite are represented as normalized stacked bars. Representative of three biological replicates. No statistical differences (P>0.05) were determined by unpaired two-sided Student's t-test. FIG. 9D, Expression of genes involved in fatty acid oxidation. RT-qPCR analysis of Cpt1a, Acadm and Hadha expression in control PMNs and PMN-MDSCs isolated from the spleen and tumors of tumor-bearing mice. Each group included 3-6 mice. Data are mean f s.d.

FIG. 10 shows the exchange of nutrients with the media. Ex vivo MDSCs were cultured in physiological-like medium supplemented with GM-CSF for 18 h. Metabolites were then extracted from the media and analysed by LC-MS. Upward bars represent efflux from the cells into the media, and downward bars represent uptake (or depletion) from the media by the cells. Data are normalized to protein content after extraction. Data are mean±s.d. (n=3).

FIG. 11A-FIG. 11J demonstrate the effect of AA on PGE2 production and suppressive activity of PMN-MDSC. FIG. 11A, LC-MS analysis of PGE₂ in PMNs from control mice and PMN-MDSCs from EL4 and CT26 tumor-bearing mice (n=3). FIG. 11B, PGE release (measured by ELISA) by control PMNs (n=4) and PMN-MDSCs from wild-type (n=11), and Slc27a2^{-/-} (FATP2 KO) (n=8) LLC tumor-bearing mice. FIG. 11C, Expression of Ptges in PMN-MDSCs isolated form the spleen of EL4 (n=13-15), KPC (n=3) and RET melanoma (n=3-6) tumor-bearing mice. FIG. 11D, Expression of Ptgs2 and Ptges (measured by qRT-PCR) in PMN-MDSCs (n=6). FIG. 11E, Expression of Arg1 and Nos2 (measured by qRT-PCR) in spleen PMN-MDSCs from wild-type and FATP2 KO EL4 tumor-bearing mice (n=3-5). FIG. 11F, Flow cytometry of myeloid cells differentiated from HPCs cultured in the presence of AA. Representative of three experiments. FIG. 11G, Expression of Arg1, Nos2 and Nox2 in PMNs isolated from HPCs cultured in the presence of AA. Data are pooled from six independent experiments. FIG. 11H, pSTAT5 expression by flow cytometry at different time points in PMNs isolated from mouse bone marrow treated with different amounts of GM-CSF. Representative of three independent experiments. FIG. 11I, LLC tumor growth (n=4) in Slc27a^{fl/fl}×S100a8-cre⁻ (Cre⁻) and Slc27a^{fl/fl}×S100a8-cre⁺ (Cre⁺) mice. FIG. 11J, Slc27a2 expression (measured by RT-qPCR) in PMN-MDSCs from the spleen of wild-type and knockout tumor-bearing mice (n=4). *P<0.05, **P<0.01, unpaired two-sided Student's t-test. Data are mean±s.d.

FIGS. 12A-FIG. 12H demonstrate lipid accumulation in MDSC from cancer patients. FIG. 12A, Lipid accumulation (measured by BODIPY staining) in M-MDSCs isolated from the blood of patients with cancer or healthy individuals. Each circle indicates an individual. FIG. 12B, Amount of lipids (BODIPY staining) in M-MDSCs from blood and tumor tissue of patients with cancer. Each circle indicates an individual (n=5). FIG. 12C, RNA-seq analysis of genes involved in lipid accumulation in human LOX1⁺ PMN-MDSCs and LOX1⁻ PMNs (n=4). FIG. 12D, PTGES expression in LOX1⁺ and LOX1⁻ PMNs from blood of patients with cancer. Fold change compared with LOX1⁻ PMNs (n=3). FIG. 12E, SLC27A2 expression in M-MDSCs and monocytes isolated from blood of patients with cancer and healthy donors, respectively. Each circle indicates an individual (n=4-6). Data are mean±s.d. FIG. 12F, pSTAT5 by flow cytometry at different time points, in human PMNs isolated from the blood of healthy donors and treated with different amounts of GM-CSF. FIG. 12G, FATP2 in PMNs isolated from blood of healthy donors and treated with GM-CSF. Representative of three independent experiments. FIG. 12H, Content of total phosphatidylethanolamine (PE) and arachidonoyl-containing phosphatidylethanolamine (AA-PE) in PMN-MDSCs isolated from patients with lung cancer (n=5) or healthy donors (n=4). Each circle indicates an individual. Data are mean±s.d. *P<0.05, **P<0.01, ***P<0.001, ****P<0.0001, unpaired two-sided Student's t-test.

FIGS. 13A-FIG. 13E show the effect of lipofermata treatment on tumor-bearing mice. FIG. 13A, MTT assay after three-day incubation of tumor cells with indicated concentration of lipofermata. FIG. 13B, Percentage and absolute number of tumor-associated antigen (E7-derived peptide)-specific CD8⁺ T cells in draining lymph nodes of mice bearing TC-1 tumor and treated with lipofermata (n=3). FIG. 13C, Growth of TC-1 tumors in mice treated with CTLA4 antibody and lipofermata (n=5). FIG. 13D, CD8⁺ T cell infiltration of TC-1 tumors in mice treated with CTLA4 antibody and lipofermata. FIG. 13E, Growth of TC-1 tumors in mice treated with PD1 antibody and lipofermata (n=5). Data are mean±s.d. *P<0.05, **P<0.01, ***P<0.001, ****P<0.0001, two-sided Student's t-test (FIG. 13B) or two-way ANOVA test with correction for repeated measurements (FIG. 13E).

FIG. 14 is a table showing major phospholipid molecular species, containing deuterated arachidonic acid in WT and FATP2 KO PMN. Phospholipid molecular species are represented as di-acyl and alkenyl/acyl species: PE(16:0/20:4) and PE(16:0p/20:4), respectively. These m/z values indicate ratios of mass to charge [M+Formate]− ions for PC and [M-H]− ions for the rest of phospholipids PE, PI, PS, PG, BMP, PA, respectively. Data are mean±SD; differences were detected by Student's t-test. N=5.

### DETAILED DESCRIPTION OF THE INVENTION

As disclosed herein, methods and compositions are described which are useful in treatment of cancer in a mammalian subject. As disclosed herein it is demonstrated that up-regulation of fatty acid transporter 2 (FATP2) is associated exclusively with PMN-MDSC; FATP2 contributes to uptake of arachidonic acid (AA) and synthesis of PGE2, which mediate PMN-MDSC suppression; deletion of FATP2 abrogated suppressive activity of PMN-MDSC and had potent antitumor effect; and targeting of FATP2 has activity as a single agent and is more potent in combination with checkpoint inhibitors.

Polymorphonuclear myeloid derived suppressor cells (PMN-MDSC) are pathologically activated neutrophils that are critically important for the regulation of immune responses in cancer. They contribute to the failure of cancer therapies and are associated with poor clinical outcomes. Despite the recent advances in understanding of the PMN-MDSC biology, the mechanisms responsible for pathological activation of neutrophils are not well defined, which limits selective targeting of these cells. Here, we report that mouse and human PMN-MDSC exclusively up-regulate fatty acid transporter protein 2 (FATP2). Over-expression of FATP2 in PMN-MDSC was controlled by GM-CSF, through the activation of STAT5 transcription factor. Deletion of FATP2 abrogated the suppressive activity of PMN-MDSC. The main mechanism of FATP2 mediated suppressive activity involved uptake of arachidonic acid (AA) and synthesis of prostaglandin E2 (PGE2). The selective pharmacological inhibition of FATP2 abrogated the activity of PMN-MDSC and substantially delayed tumor progression. In combination with check-point inhibitors, inhibition of FATP2 blocked tumor progression in mice. Thus, FATP2 mediates acquisition of immune suppressive activity by PMN-MDSC and represents a new target to selectively inhibit the functions of PMN-MDSC and improve the effect of cancer therapy.

MDSC have been divided in two large sub-populations, monocytic myeloid-derived suppressor cells (M-MDSC) and polymorphonuclear myeloid-derived suppressor cells (PMN-MDSC). About 20-30% of MDSC consists of monocytic cells, i.e., M-MDSC, and are generally associated with high activity of Arginase-1 and iNOS. Two different phenotypes (CD11b⁺ CD14⁻ CD15⁻ and CD33⁺ or CD11b⁺ CD14⁺ CD33⁺ and HLA-DR^{lo}) are used to characterize these M-MDSC cells depending on the type of cancer.

The second population, i.e., PMN-MDSC, are comprised of granulocytic cells and are usually associated with high level of ROS production. PMN-MDSC represent the major population of MDSC (about 60-80%) and represent the most abundant population of MDSC in most types of cancer. PMN-MDSC are phenotypically and morphologically similar to neutrophils (PMN) and share the CD11b+CD14−CD15+/CD66b+ phenotype. The may also be characterized as CD33⁺. PMN-MDSC are important regulators of immune responses in cancer and have been directly implicated in promotion of tumor progression.

### Definitions and Components of the Methods

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

“Patient” or “subject” as used herein means a mammalian animal, including a human, a veterinary or farm animal, a domestic animal or pet, and animals normally used for clinical research. In one embodiment, the subject of these methods and compositions is a human.

The term “fatty acid transporter 2”, “FATP2”, also termed Very long-chain acyl-CoA synthetase, as used herein refers to fatty acid transport protein 2, encoded by the SLC27A2 gene, or isoforms thereof. The amino acid sequence of FATP2 is known in the art (UniProtKB-O14975) and set forth below:

(SED ID NO: 1)
MLSAIYTVLA GLLFLPLLVN LCCPYFFQDI GYFLKVAAVG
RRVRSYGKRR PARTILRAFL EKARQTPHKP FLLFRDETLT
YAQVDRRSNQ VARALHDHLG LRQGDCVALL MGNEPAYVWL
WLGLVKLGCA MACLNYNIRA KSLLHCFQCC GAKVLLVSPE
LQAAVEEILP SLKKDDVSIY YVSRTSNTDG IDSFLDKVDE
VSTEPIPESW RSEVTFSTPA LYIYTSGTTG LPKAAMITHQ
RIWYGTGLTF VSGLKADDVI YITLPFYHSA ALLIGIHGCI
VAGATLALRT KFSASQFWDD CRKYNVTVIQ YIGELLRYLC
NSPQKPNDRD HKVRLALGNG LRGDVWRQFV KRFGDICIYE
FYAATEGNIG FMNYARKVGA VGRVNYLQKK IITYDLIKYD
VEKDEPVRDE NGYCVRVPKG EVGLLVCKIT QLTPFNGYAG
AKAQTEKKKL RDVFKKGDLY FNSGDLLMVD HENFIYFHDR
VGDTFRWKGE NVATTEVADT VGLVDFVQEV NVYGVHVPDH
EGRIGMASIK MKENHEFDGK KLFQHIADYL PSYARPRFLR
IQDTIEITGT FKHRKMTLVE EGFNPAVIKD ALYFLDDTAK
MYVPMTEDIY NAISAKTLKL

The nucleic acid sequence for the gene encoding FATP2 (gene name SLC27A2) can be found in databases such as GenBank: KJ893197.1. It should be understood that the term FATP2 can also represent the protein in various species, and with conservative changes in the amino acid or encoding sequences, or with other naturally occurring modifications that may vary among species and between members of the same species, as well as naturally occurring mutations thereof.

The term “cancer” or “tumor” as used herein refers to, without limitation, refers to or describes the physiological condition in mammals that is typically characterized by unregulated cell growth. Cancer as used herein is meant any form of cancer, including hematological cancers, e.g., leukemia, lymphoma, myeloma, bone marrow cancer, and epithelial cancers, including, without limitation, breast cancer, lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, melanoma, leukemia, myeloma, lymphoma, glioma, Non-Hodgkin's lymphoma, leukemia, multiple myeloma and multidrug resistant cancer. In one embodiment, the cancer is lymphoma. In another embodiment, the cancer is carcinoma. In another embodiment, the cancer is myeloma.

A “tumor” is an abnormal mass of tissue that results from excessive cell division that is uncontrolled and progressive, and is also referred to as a neoplasm. The term “tumor,” as used herein, refers to all neoplastic cell growth and proliferation, whether malignant or benign, and all pre-cancerous and cancerous cells and tissues.

As used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing, inhibiting, or reducing the extent of metastasis, and/or extending the survival of an animal having cancer compared to control.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

The term “myeloid-derived suppressor cells” or “MDSCs” refers to cells of myeloid origin that are undergo expansion in a cancer-related context and have been described as having certain immune-suppressive functions. MDSC comprise two major subsets termed polymorphonuclear (PMN) and monocytic (M)-MDSC, also PMN-MDSC and M-MDSC, respectively. PMN-MDSC and M-MDSC are phenotypically and morphologically distinct, but share some overlapping functional characteristics and biochemical traits. In certain embodiments, PMN-MDSCs and M-MDSCs are identified or separated using markers including, for example, CD11b, CD14, CD15, CD66, HLA-DR, CD33. In human peripheral blood, for example, PMN-MDSC can be identified as CD11b⁺ CD14⁻ CD15⁺ or CD11b⁺ CD14⁻ CD66b⁺ and M-MDSC as CD11b⁺ CD14⁺HLA-DR^{-/lo}CD15⁻. In certain embodiments, lectin-type oxidized LDL receptor 1 (LOX-1) is used as a marker to identify PMN-MDSCs in a sample. In certain embodiments, LOX-1 expression distinguishes PMN-MDSCs from, for example, neutrophils or PM-LCs in a sample. In some embodiments, PMN-MDSCs are identified based on the expression or level of expression of certain genes that make up a gene signature, which may include one or more of HLA-DPA1, HLA-DRA, EBI2, OLR1 THBS1, CD36, MMD, ASGR1, CD69, HLA-DRB3, CD74, RPSA, HLA-DQA1, CD86, PTGER2, ITGB5, CD79B, CD79A, IL10RA, PLXNB2, ITGB1, PLAUR, CD247, SCARB2, CD1D, GPBAR1, CLEC1B, TFRC, ITGB3, CD300C, ITGA22B and CXCR5. In certain embodiments, MDSCs are identified based on expression of one or more of MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, or Fin, CD15, CD66b or CD33. Suitable markers and functional assays for identifying or distinguishing MDSC subsets are described in the art (See, for example, Bronte, V. et al. Recommendations for myeloid-derived suppressor cell nomenclature and characterization standards. Nat. Commun. 7: 12150 (2016), which is incorporated herein by reference).

As used herein for the described methods and compositions, the term “antibody” refers to an intact immunoglobulin having two light and two heavy chains or fragments thereof capable of binding to a biomarker protein or a fragment of a biomarker protein. Thus, a single isolated antibody or an antigen-binding fragment thereof may be a monoclonal antibody, a synthetic antibody, a recombinant antibody, a chimeric antibody, a humanized antibody, a human antibody, or a bi-specific antibody or multi-specific construct that can bind two or more target biomarkers.

The term “antibody fragment” as used herein for the described methods and compositions refers to less than an intact antibody structure having antigen-binding ability. Such fragments, include, without limitation, an isolated single antibody chain or an scFv fragment, which is a recombinant molecule in which the variable regions of light and heavy immunoglobulin chains encoding antigen-binding domains are engineered into a single polypeptide. Other scFV constructs include diabodies, i.e., paired scFvs or non-covalent dimers of scFvs that bind to one another through complementary regions to form bivalent molecules. Still other scFV constructs include complementary scFvs produced as a single chain (tandem scFvs) or bispecific tandem scFvs.

Other antibody fragments include an FIT construct, a Fab construct, an Fc construct, a light chain or heavy chain variable or complementarity determining region (CDR) sequence, etc. Still other antibody fragments include monovalent or bivalent minibodies (miniaturized monoclonal antibodies) which are monoclonal antibodies from which the domains non-essential to function have been removed. In one embodiment, a minibody is composed of a single-chain molecule containing one V_{L}, one V_{H} antigen-binding domain, and one or two constant “effector” domains. These elements are connected by linker domains. In still another embodiment, the antibody fragments useful in the methods and compositions herein are “unibodies”, which are IgG4 molecules from with the hinge region has been removed.

The antibodies of the instant invention may also be conjugated/linked to other components. For example, the antibodies may be operably linked (e.g., covalently linked, optionally, through a linker) to at least one cell penetrating peptide, detectable agent, imaging agent, or contrast agent. The antibodies of the instant invention may also comprise at least one purification tag (e.g., a His-tag). In a particular embodiment, the antibody is conjugated to a cell penetrating peptide.

Arachidonic acid is an unsaturated, essential fatty acid. It is found in animal and human fat as well as in the liver, brain, and glandular organs, and is a constituent of animal phosphatides. It is formed by the synthesis from dietary linoleic acid and is a precursor in the biosynthesis of prostaglandins, thromboxanes, and leukotrienes. The COX-2 enzyme catalyzes conversion of arachidonic acid to different prostaglandins such as prostaglandin E2 (PGE2).

The terms “analog”, “modification” and “derivative” refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined herein. Preferably, the analog, modification or derivative has at least the same desired activity as the native molecule, although not necessarily at the same level. The terms also encompass purposeful mutations that are made to the reference molecule. Particularly preferred modifications include substitutions that are conservative in nature, i.e., those substitutions that take place within a family of amino acids that are related in their side chains. Specifically, amino acids are generally divided into four families: acidic, basic, non-polar and uncharged polar. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-20 conservative or non-conservative amino acid substitutions, so long as the desired function of the molecule remains intact. One of skill in the art can readily determine regions of the molecule of interest that can tolerate change by reference to Hopp/Woods and Kyte Doolittle plots, well known in the art.

One skilled in the art may readily reproduce the compositions and methods described herein by use of the amino acid sequences of the biomarkers and other molecular forms, which are publicly available from conventional sources.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

Provided herein are methods and compositions for, inter alia, treating cancer in a subject in need thereof. In one embodiment, the method includes administering an effective amount of an agent that inhibits, decreases, deletes, or downregulates FATP2 in the subject. In another aspect, a method of decreasing MDSCs in a subject in need thereof is provided which includes inhibiting, decreasing, deleting, or downregulating FATP2 in the subject. In yet another aspect, a method of decreasing the immune-suppressive activity of PMN-MDSCs in a subject in need thereof is provided which includes inhibiting, decreasing, deleting, or downregulating FATP2 in the subject. Such agents are described herein.

Inhibitors of FATP2 have been implicated for having potential therapeutic applicability for the prevention and resolution of diseases involving fatty acid uptake and metabolism including but not limited to obesity, Type 2 Diabetes, metabolic syndrome, cardiovascular disease, and non-alcoholic fatty liver disease. However, it is believed that the instant disclosure is the first to provide evidence for the use of these compounds in the reduction of MDSC and treatment of cancer.

The methods described herein utilize compositions which include agents which inhibit, decrease, delete, or downregulate FATP2 in the subject. In some embodiments, such agents are known in the art. In one embodiment, the agent which inhibits FATP2 is 5′-bromo-5-phenyl-spiro[3H-1,3,4-thiadiazole-2,3′-indoline]-2′-one), referred to herein as lipofermata. In another embodiment, the agent which inhibits FATP2 is 2-benzyl-3-(4-chlorophenyl)-5-(4-nitrophenyl)pyrazolo[1,5-a]pyrimidin-7(4H)-one, referred to herein as grassofermata. See, Black et al, Fatty Acid Transport Proteins: Targeting FATP2 as a Gatekeeper Involved in the Transport of Exogenous Fatty Acids, Medchemcomm. 2016 Apr. 1; 7(4): 612-622, which is incorporated herein by reference. Other compounds known to inhibit FATP2 are known in the art and include those described in U.S. Pat. No. 8,263,640B2 and U.S. Pat. No. 8,431,582, which are incorporated herein by reference.

Another inhibitor of FATP2 useful herein is (5E)-5-((3-bromo-4-hydroxy-5-methoxyphenyl)methylene)-3-(3-chlorophenyl)-2-thioxothiazolidin-4-one, also called CB2. See, Sandoval et al, Identification and characterization of small compound inhibitors of human FATP2, Biochem Pharmacol. 2010 Apr. 1; 79(7): 990, which is incorporated herein by reference.

In a particular embodiment, the inhibitor is a small molecule inhibitor of FATP2. In a particular embodiment, the inhibitor is an inhibitory nucleic acid molecule (e.g., antisense, siRNA, shRNA, etc.) or a vector encoding the same. In a certain embodiments, the inhibitor is an antibody or antibody fragment that is immunologically specific for FATP2. (e.g., a neutralizing antibody).

In certain embodiments, an agent that inhibits, decreases, deletes, or downregulates FATP2 is administered in combination with a composition that inhibits fat absorption (e.g. Xenical (orlistat)).

As used herein, the term “small molecule” refers to a substance or compound that has a relatively low molecular weight (e.g., less than 2,000). Typically, small molecules are organic, but are not proteins, polypeptides, or nucleic acids.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al. (2006) Current Protocols in Molecular Biology, John Wiley and Sons, Inc). As used herein, the term siRNA may include short hairpin RNA molecules (shRNA). Typically, shRNA molecules consist of short complementary sequences separated by a small loop sequence wherein one of the sequences is complimentary to the gene target. shRNA molecules are typically processed into an siRNA within the cell by endonucleases. Exemplary modifications to siRNA molecules are provided in U.S. Application Publication No. 20050032733. Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502 09).

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

A “cell-penetrating peptide” refers to a peptide which can transduce another peptide, protein, or nucleic acid into a cell in vitro and/or in vivo—i.e., it facilitates the cellular uptake of molecules. Examples of cell penetrating peptides include, without limitation, Tat peptides, penetratin, transportan, and the like.

The term “therapeutically effective amount” or “effective amount” refers to an amount agent that when administered alone or in combination with an additional therapeutic agent to a cell, tissue, or subject is effective to prevent or ameliorate the tumor-associated disease condition or the progression of the disease, e.g., metastasis. A therapeutically effective dose further refers to that amount of the compound sufficient to result reduction, prevention or inhibition of metastasis. For example, when in vivo administration of an agent is employed, normal dosage amounts may vary from about 10 ng/kg to up to 100 mg/kg of subject body weight or more per dosage or per day, preferably about 1 μg/kg to 50 mg/kg, optionally about 100 μg/kg to 20 mg/kg, 500 μg/kg to 10 mg/kg, or 1 mg/kg to 10 mg/kg, depending upon the route of administration.

A reduction or inhibition of cancer can be measured relative to the incidence observed in the absence of the treatment and, in further testing, inhibits tumor growth. The tumor inhibition can be quantified using any convenient method of measurement. Tumor growth can be reduced by about 10%, 20%, 30%, 40%, 50%, 60%, 70%, 80%, 90%, 95% or greater.

Other therapeutic benefits or beneficial effects provided by the methods described herein may be objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Also provided herein are methods of decreasing arachidonic acid (AA) or arachidonic acid-containing phospholipids in a subject in need thereof by administering an inhibitor of FATP2. AA is metabolized to PGE2 (amongst other prostaglandins) which promotes PMN-MDSC generation and suppression. In another embodiment, a method is provided where PGE synthesis is decreased by administering a FATP2 inhibitor.

Also provided herein are methods and compositions that include one or more agents which inhibits FATP2 in combination with one or more additional therapeutic agent(s). Such compositions include all inhibitors of FATP2 including, without limitation, peptides, nucleic acid molecules, small molecule compounds, antibodies and derivatives thereof. Also provided herein are pharmaceutical compositions that include FATP2 inhibitors optionally in combination with one or more additional therapeutic agent(s).

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments; the additional therapeutic agent is an anti-LAG-3 antibody selected from the group consisting of: BMS-986016 and LAG525. In some embodiments, the additional therapeutic agent is an anti-OX-40 antibody selected from: MEDI6469, MEDI0562, and MOXR0916. In some embodiments, the additional therapeutic agent is the anti-4-1BB antibody PF-05082566.

The present disclosure provides compositions and methods that include blockade of immune checkpoints. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (AZAR), B7-H3 (also known as CD276); B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

It is contemplated that any of the immune checkpoint inhibitors that are known in the art to stimulate immune responses may be used. This includes inhibitors that directly or indirectly stimulate or enhance antigen-specific T-lymphocytes. These immune checkpoint inhibitors include, without limitation, agents targeting immune checkpoint proteins and pathways involving PD-L2, LAG3, BTLA, B7H4 and TIM3. For example, LAG3 inhibitors known in the art include soluble LAG3 (IMP321, or LAG3-Ig disclosed in WO2009044273) as well as mouse or humanized antibodies blocking human LAG3 (e.g., IMP701 disclosed in WO2008132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO2011014438). Yet another example is provided by the use of agents neutralizing B7H4 including without limitation antibodies to human B7H4 (disclosed in WO 2013025779, and in WO2013067492) or soluble recombinant forms of B7H4 (such as disclosed in US20120177645). Yet another example is provided by agents neutralizing B7-H3, including without limitation antibodies neutralizing human B7-H3 (e.g. MGA271 disclosed as BRCA84D and derivatives in US 20120294796). Yet another example is provided by agents targeting TIM3, including without limitation antibodies targeting human TIM3 (e.g. as disclosed in WO 2013006490 A2 or the anti-human TIM3, blocking antibody F38-2E2 disclosed by Jones et al., J Exp Med. 2008; 205(12):2763-79).

In addition, more than one immune checkpoint inhibitor (e.g., anti-PD-1 antibody and anti-CTLA-4 antibody) may be used in combination with the FATP2 inhibitor. For example, p53 gene therapy and immune checkpoint inhibitors (e.g., anti-MR antibody and/or anti-PD-1 antibody) can be administered to enhance innate anti-tumor immunity followed by IL24 gene therapy and immune checkpoint inhibitors (e.g., anti-PD-1 antibody) to induce adaptive anti-tumor immune responses.

Provided herein is a method for treating or delaying progression of cancer in an individual comprising administering to the individual an effective amount of a PD-1 axis binding antagonist in combination with a FATP2 inhibitor. For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesion, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 binding antagonists include Pidilizumab, also known as CT-011, MEDI0680, also known as AMP-514, and REGN2810.

In some aspects, the immune checkpoint inhibitor is a PD-L1 antagonist such as Durvalumab, also known as MEDI4736, atezolizumab, also known as MPDL3280A, or avelumab, also known as MSB00010118C. In certain aspects, the immune checkpoint inhibitor is a PD-L2 antagonist such as rHIgM12B7. In some aspects, the immune checkpoint inhibitor is a LAG-3 antagonist such as, but not limited to, IMP321, and BMS-986016. The immune checkpoint inhibitor may be an adenosine A2a receptor (A2aR) antagonist such as PBF-509.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006, CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

Also provided herein are methods and compositions that include one or more agents which inhibits FATP2 in combination with a CSF-1/1R binding agent or inhibitor (e.g. an anti-CSF1 or anti-CSF1R antibody), where the combination is used to treat a cancer, e.g., a cancer described herein, e.g., a solid tumor. In certain embodiments, the CSF-1/1R binding agent is a CSF-1R tyrosine kinase inhibitor, 4-((2-(((1R,2R)-2-hydroxycyclohexyl)amino)benzo[d]thiazol-6-yl)oxy)-N-met-hylpicolinamide (Compound A15), or a compound disclosed in PCT Publication No. WO 2005/073224. In certain embodiments, the CSF-1/1R binding agent is an M-CSF inhibitor, Compound A33, or a binding agent to CSF-1 disclosed in PCT Publication No. WO 2004/045532 or PCT Publication No WO 2005/068503 including RX 1 or 5H4 (e.g., an antibody molecule or Fab fragment against M-CSF). In certain embodiments, the CSF-1/1R binding agent is 4-(2-((1R, 2R)-2-hydroxycyclohexylamino)benzothiazol-6-yloxy)-N-methylpicolinamide, or BLZ-945. 4-(2-((1R, 2R)-2-hydroxycyclohexylamino)benzothiazol-6-yloxy)-N-methylpicolinamide is disclosed as example 157 at page 117 of PCT Publication No. WO 2007/121484. In certain embodiments, the CSF-1/1R binding agent is pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromet-hyl)pyridin-3-yl)methyl)pyridin-2-amine. Pexidartinib is a small-molecule receptor tyrosine kinase (RTK) inhibitor of KIT, CSF1R and FLT3. In certain embodiments, the CSF-1/1R binding agent is emactuzumab. Emactuzumab is also known as RG7155 or R05509554. Emactuzumab is a humanized IgG1 mAb targeting CSF1R. In certain embodiments, the CSF-1/1R binding agent is FPA008. FPA008 is a humanized mAb that inhibits CSF1R.

Various routes of administration are useful in these methods. In one embodiment, the composition is delivered to the tumor site itself. In another embodiment, the composition is administered IV.

Pharmaceutical compositions may be formulated for any appropriate route of administration. For example, compositions may be formulated for intravenous, parenteral, subcutaneous, intramuscular, intracranial, intraorbital, ophthalmic, intraventricular, intracapsular, intraspinal, intracisteral, intraperitoneal, intranasal, or aerosol administration. In some embodiments, pharmaceutical compositions are formulated for direct delivery to the tumor (intratumoral) or to the tumor environment. In another embodiment, pharmaceutical compositions are formulated for delivery to the lymph nodes.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc. Methods and agents well known in the art for making formulations are described, for example, in “Remington's Pharmaceutical Sciences,” Mack Publishing Company, Easton, Pa. Formulations may, for example, contain excipients, diluents such as sterile water, or saline, polyalkylene glycols such as polyethylene glycol, oils of vegetable origin, or hydrogenated napthalenes.

In another aspect, a pharmaceutical composition comprises the FATP2 inhibitor(s) described above optionally in combination with a checkpoint inhibitor in a pharmaceutically acceptable carrier or excipient in an effective amount to reduce, inhibit, retain or suppress growth of MDSC and/or the PMN-MDSC population. In yet another aspect, a pharmaceutical composition comprises the FATP2 inhibitor(s) described above optionally in combination with a checkpoint inhibitor in a pharmaceutically acceptable carrier or excipient in an effective amount to reduce, inhibit, retain or suppress tumor growth or treat cancer. In one aspect, the pharmaceutical composition contains, e.g., about 25, 30, 35, 40, 45, 50, 55, 60, 65, 70, 75, 80, 85, to about 90% of the antagonist or inhibitors in combination with a pharmaceutical carrier or excipient.

By “pharmaceutically acceptable carrier or excipient” is meant a solid and/or liquid carrier, in in dry or liquid form and pharmaceutically acceptable. The compositions are typically sterile solutions or suspensions. Examples of excipients which may be combined with the antagonist or inhibitor include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanth, acacia, starch, gelatin, polyglycolic acid, polylactic acid, poly-d,l-lactide/glycolide, polyoxaethylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, D.C.), 2005 and U.S. Pat. No. 7,078,053, which are incorporated herein by reference. The selection of the particular excipient is dependent on the nature of the compound selected and the particular form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide (DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

In one embodiment, the effective amount of the inhibitors is within the range of 1 mg/kg body weight to 100 mg/kg body weight in humans including all integers or fractional amounts within the range. In certain embodiments, the effective amount is at least 2, 5, 10, 15, 20, 25, 30, 35, 40, 45, 50, 55, 60, 65, 70, 75, 80, 85, 90, 95 or 100 mg/kg body weight, including all integers or fractional amounts within the range. In one embodiment, the above amounts represent a single dose. In another embodiment, the above amounts define an amount delivered to the subject per day. In another embodiment, the above amounts define an amount delivered to the subject per day in multiple doses. In still other embodiments, these amounts represent the amount delivered to the subject over more than a single day.

In another embodiment, the pharmaceutical composition includes a FATP2 inhibitor and a chemotherapeutic. Alternatively, the active compound is formulated with a chemotherapeutic for treatment of the cancers described herein. In one embodiment, the chemotherapeutic is selected from among those described above. Alternatively, the composition is formulated with another effective compound or reagent for treatment of the cancers described herein, such as an antibiotic or bactericide, a surfactant, or other reagent commonly used in formulation of anti-cancer compositions.

The forms of the pharmaceutical compositions may be liquid, solid or a suspension or semi-solid and designed for use with a desired administrative route, such as those described herein. The doses and dosage regimens are adjusted for the particular cancer, and the stage of the cancer, physical status of the subject. Such doses may range from about 1 to about 100 mg/kg subject body weight of the antagonist or inhibitor, as discussed above and include dosage regimens designed to administer the effective amount in smaller repeated doses.

Polymorphonuclear myeloid derived suppressor cells (PMN-MDSC) are pathologically activated neutrophils that accumulate in many diseases. These cells are critically important for the regulation of immune responses in cancer, promotion of tumor progression, and metastases, and their presence correlates with poor prognosis and negative response to immunotherapy¹⁻⁴. Despite the fact that neutrophils and PMN-MDSC share same origin and the same differentiation pathways, PMN-MDSC have distinct genomic and biochemical features and are immunosuppressive². The mechanisms responsible for pathological activation of neutrophils are not well defined, which limits selective targeting of these cells. Here, we report a specific role of the fatty acid (FA) transport protein 2 (FATP2) in regulation of PMN-MDSC function.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### EXAMPLES

### Example 1: Materials and Methods

### Human Samples

Samples of peripheral blood and tumor tissues were collected from patients at Helen F. Graham Cancer Center and University of Pennsylvania. The study was approved by Institutional Review Boards of the Christiana Care Health System at the Helen F. Graham Cancer Center, and The Wistar Institutional Review Board. All patients signed IRB approved consent forms. Samples were collected at Helen F. Graham Cancer Center from 6 patients with previously untreated stage II-IV non-small cell lung cancer (NSCLC), 11 patients with stage III-IV head and neck cancer and 5 patients with stage III-IV breast cancer. This cohort includes 12 females and 10 males, aged 48-74 years. Peripheral blood was also collected from 9 healthy volunteers after obtaining informed consent.

### Mouse Models

Animal experiments were approved by The Wistar Institute Animal Care and Use Committee. Balb/c or C57BL/6 mice (female, 6-8 week old) were obtained from Charles River, OT-I TCR-transgenic mice (C57Bl/6-Tg(TCRaTCRb)1100mjb) (female, 6-8 week old), B6.12951-Cd36tm1Mfe/J, B6.Cg-Msr1tm1Csk/J, 129S-Slc27a2tm1Kds/J were purchased from Jackson Laboratory. C57Bl/6-Slc27a2tm1Kds/J were generated by backcrossing 129S-Slc27a2tm1Kds/J with wild type C57Bl/6 for ten generations. B6.129S6-Stat5^{btm1Mam} Stat5atm2^{Mam/Mmjax} were crossed with B6.Cg-Tg(S100A8-cre,-EGFP) 1Ilw/J, obtained from Jackson Laboratory. RET melanoma were obtained from Dr. Umansky (German Cancer Center, Heidelberg, Germany). Sc127a4^{fl/fl} were obtained from Dr. Stremmel (University of Heidelberg, Germany) and crossed with B6.Cg-Tg(S100A8-cre,-EGFP) 1Ilw/J (Jackson Laboratory). In mouse tumor models maximal tumor size approved by IACUC was 2 cm in larger diameter. In none of the experiments were these limits exceeded. Sample size calculation was performed in advance. Studies were not blinded. In treatment experiments, mice were randomized prior to start of therapy to different groups based on equal tumor size.

### Generation of PMN-Specific SLC27A2-Deficient Mice

Mice were generated at CRISPR/Cas9 Mouse Targeting Core of University of Pennsylvania, using the CRISPR/Cas9 system as described³¹. Conditional knock-out mice (CKO Sc127a2^{fl/fl}) were generated using CRISPR-Cas9 genome-editing system, at CRISPR Cas9 Mouse Targeting Core of University of Pennsylvania by flanking Exon 1 with loxp sites, as described³¹. The sequences for the gRNAs and repair templates used are as follows:

Slc27a2 5′gRNA
(SEQ ID NO: 2):
GTCCACAATACCGTCGATGTGTTTTAGAGCTAGAAATAGCAAGTTAAAAT
AAGGCTAGTCCGTTATCAACTTGAAAAAGTGGCACCGAGTCGGTGCTTTT
TT
Slc27a2 3′ gRNA
(SEQ ID NO: 3):
ACTCCTCCGTTATATGATTGGTTTTAGAGCTAGAAATAGCAAGTTAAAAT
AAGGCTAGTCCGTTATCAACTTGAAAAAGTGGCACCGAGTCGGTGCTTTT
TT
Slc27a2 5′ LoxP oligoDNA
(SEQ ID NO: 4):
TTTACTTTGTTTGCTTTGTGTGTTTTGGTAAATGTCGAACTGAGTCCACA
ATACCGTCGATGTataacttcgtataatgtatgctatacgaagttatTGG
AAAGTGGCTCGCGTAACAGAACAAAATCTCAAAACAAATTAACAGGACCC
CATTGCTCGA
Slc27a2 3′ LoxP oligoDNA
(SEQ ID NO: 5):
ATACTGTAATGGATGGTTTTAATATTCTGATAAATTAAAAATCACTCCTC
CGTTATATGATTGataacttcgtataatgtatgctatacgaagttatAGG
AAACATATAGAATTTTCCAGCCTAGCTCCGTCTTCAAAGCCCACGTTTCT
TATACAGTGC

Sc127a2^{fl/fl} mice were then crossed with B6.Cg-Tg(S100A8-cre,-EGFP) 1Ilw/J (Jackson Laboratory) to obtain mice with targeted deletion of FATP2.

### Reagents and Cell Lines

Tumor cell lines: EL4 (lymphoma), LLC (Lewis Lung Carcinoma), CT26 (colon carcinoma), TC-1 (HPV16 E6/E7 expressing tumor cell line) were obtained from ATCC and F244 (sarcoma) was kindly provided by Dr. R. Schreiber (Washington University, St. Lois, Mo.). All cells were maintained in DMEM medium supplemented with 10% fetal bovine serum (FBS, Sigma-Aldrich, St. Louis, Mo.) at 37° C., 5% CO₂. Tumor cells were injected subcutaneously (s.c.) at 5×10⁵ cells per mouse. Tumor cell lines were obtained from ATCC and were tested for Mycoplasma contamination by using Universal Mycoplasma detection kit (ATCC) every 3 months. SIINFEKL and EGSRNQDWL peptides were obtained from American Peptide Company (Vista, Calif.).

### Preparation of TES

Tumor explant supernatants (TES) were prepared from excised non-ulcerated EL4 tumors ˜1.5 cm in diameter. A small tumor piece (5-10 mm²) was harvested, minced into pieces <3 mm in diameter and resuspended in complete RPMI without extra cytokines. After 16-18 hours of incubation at 37° C. with 5% CO₂, the cell-free supernatant was collected using 0.22 μm filters (EMD Millipore) and kept at −80° C.

### Cell Phenotype, Lipid Contents by Flow Cytometry and by Confocal Microscopy

Cells were incubated with FC-block (BD Biosciences) for 10 min and surface staining was performed at 4° C. for 15 min. Cells were run on LSRII flow cytometer (BD Biosciences) and data were analyzed by FlowJo (Tristar). For lipid staining by flow cytometry, cells were re-suspended in 500 μl of Bodipy 493/503 at 0.25 μg/ml in PBS. Cells were stained for 15 min at room temperature in the dark, then washed twice, re-suspended in PBS and run immediately on LSRII. For lipid staining by confocal microscopy, cells were washed twice with PBS, resuspended in complete RPMI and 50,000 cells were seeded on poly-L-lysine cellware 12 MM round coverslips (Corning) for 45 min at 37° C. Cells were fixed and permeabilized with Fixation & Permeabilization Buffers (BD Biosciences) for 15 min at RT, washed twice with wash buffer (BD Biosciences) and then stained with BODIPY for 15 min at RT. Cells were washed and incubated with DAPI and mounted on slides using Prolong Gold antifade reagent (Life Technology). The cells were imaged with a Leica TCS SP5 laser scanning confocal microscope (Leica Microsystems).

### Isolation of Mouse Cells

Single-cell suspensions were prepared from spleen and followed by red blood cell removal using ammonium chloride lysis buffer. Single-cell suspensions from tumor tissues were prepared using Mouse Tumor Dissociation Kit according to the manufacturer's recommendation (Miltenyi). CD8⁺ T cells were isolated from spleen and lymph nodes by using EasySep Mouse CD8⁺ T Cell Enrichment Kit (STEMCELL), following manufacturer's instructions.

### Suppression Assay

Single cells suspensions from spleen and tumors were prepared as described above. Then cells were stained and sorted on BD FACS Aria BD (Biosciences). PMN-MDSC)(CD45⁺ CD11b⁺Ly6G⁺Ly6C^{lo} and M-MDSC (CD45⁺ CD11b⁺Ly6G⁻Ly6C^{hi}) were plated in U-bottom 96-well plates (3 replicates) in RPMI with 10% FBS and co-cultured at different ratios with splenocytes from Pmel or OT-1 transgenic mice in the presence of cognate peptides: OT-1 (SIINFEKL; 0.1 ng/ml), Pmel (EGSRNQDWL; 0.1 μg/ml). After 48 h, cells were incubated with [³H]-thymidine (PerkinElmer) for 16-18 h. Proliferation was measured by using TopCount NXT instrument (PerkinElmer).

### IFNγ ELISpot, T Cell Proliferation and Antigen Specific T Cell Analysis

Lymph nodes (LNs) were obtained from TB mice and digested for 30 min at 37° C. with collagenase A (0.5 mg/ml; Sigma Aldrich), Dnase I (0.2 mg/ml, Roche), diluted in HBSS with Ca²⁺/Mg²⁺ and 20 mM EDTA (Invitrogen) was added 5 min at room temperature to stop the reaction. CD8⁺ T cells were isolated from LNs of TB mice using EasySep Mouse CD8+ T Cell Enrichment Kit (STEMCELL) and stimulated with anti CD3 and anti CD28 antibodies (BD Biosciences) for 24 h and IFNγ was analyzed by ELISpot (Mabtech), accordingly to manufacturer's instructions. T cell proliferation was evaluated by flow cytometry using CSFE (BioLegend). Antigen specific CD8 T cell response was evaluated in single cell suspension obtained from LNs of TC1 (HPV16 E6/E7 expressing tumor cells) TB mice by flow cytometry using MHC tetramer (H-2db HPV 16 E7-RAHYNIVTF), obtained from D. Weiner (Wistar Institute, Philadelphia, USA).

### Isolation of Human Cells

PMN-MDSC and PMN were isolated by centrifugation over a double density gradient Histopaque (Sigma) (1.077 to collect PBMC and 1.119 to collect PMN) followed by labeling with CD15-PE mAb (BD Biosciences) and then separated using anti-PE beads and MACS column (Miltenyi). Tissues were first digested with human tumor dissociation kit (Miltenyi) and then red blood cell lysed. Cells were then culture in RPMI (Biosource International) supplemented with 10% FBS, 5 mM glutamine, 25 mM HEPES, 50 μM β-mercaptoethanol and 1% antibiotics (Invitrogen). For isolation of Lox1⁺ PMN from peripheral blood, whole blood was enriched for PMNs using MACSxpress® Neutrophil Isolation Kit (Miltenyi) following the protocol provided by the manufacturer. Cells were then labeled with anti-Lox1-PE mAb (Biolegend) and then separated using anti-PE beads and MACS column (Miltenyi).

### Quantitative Real Time PCR

RNA was extracted using Total RNA Kit according to manufacturer's instructions. DNase digestion was performed cDNA was generated with High-Capacity cDNA Reverse Transcription Kit (Applied Biosystems, Foster City, Calif., USA). qRT-PCR was performed using Power SYBR Green PCR Master Mix (Applied Biosystems) in 96- or 384 well plates. Plates were read with ABI 7900 (Applied Biosystems).

### RNA-Seq

RNA-seq data was aligned using bowtie2³² against mm10 version of the mouse genome and RSEM v1.2.12 software³³ was used to estimate raw read counts using Ensemble v84 gene information. DESeq2³⁴ was used to estimate significance of differential expression between sample groups. Overall gene expression changes were considered significant if passed false discovery rate FDR<5% threshold. Significant genes affected at least 2 fold were analyzed for enrichment of upstream regulators using QIAGEN's Ingenuity® Pathway Analysis software (IPA®, QIAGEN Redwood City, www.qiagen.com/ingenuity, “Upstream Analysis” option). Only regulators with significantly enriched p<0.005 targets (at least 20) with significantly predicted activation state (activation z-score |Z|>2) were considered.

### Western Blot

Cells were lysed in RIPA buffer (Sigma-Aldrich) in presence of protease inhibitor cocktail (Sigma-Aldrich), sonicated and stored at −80° C. Whole cell lysates were prepared and subjected to 10% SDS-PAGE and transferred to PVDF membrane. The membranes were probed overnight at 4° C. with the antibodies specific for FATP2 (SLC27A2) (ThermoFisher), ACTIN, GAPDH, STAT5 (Cell Signaling Technology). Membranes were washed and incubated for 1 h at room temperature with secondary antibody conjugated with peroxidase.

### Generation of Suppressive Neutrophils In Vitro

Mouse neutrophils were generated from enriched bone marrow hematopoietic progenitor cells (HPCs) with 20 ng/ml of GM-CSF. Briefly, HPCs were isolated from mouse BM by using Lineage depletion kit (Miltenyi), according to manufacturer's instructions. Cells were seeded at 25000 cell/ml in 24 well plates and GM-CSF (20 ng/ml), 20% v/v TES or arachidonic acid (10 μM) were added at day 0 and day 3. At day 5, Ly6G positive neutrophils were isolated by using anti-Ly6G biotin (Miltenyi) and streptavidin beads (Miltenyi), according to manufacturer's instructions.

### FATP2 Overexpression in HPC and PGE2 ELISA

HPCs were isolated from mouse BM by using Lineage depletion kit (Miltenyi), according to manufacturer's instructions. HPC were resuspended in serum-free medium (SFM) containing lentiviral vectors followed by centrifugation of the plate at 1000 rpm for 20 min at 25° C. Fresh media supplemented with GM-CSF (20 ng/ml) was then added and cells were seeded at 25000 cell/ml in 24 well plates. At day 3, GM-CSF and 20% v/v TES were added to the culture. At day 5, cells were collected, stained with PE conjugated anti mouse Ly6G, APC conjugated anti mouse Ly6C and BV421 conjugated anti-mouse CD11b, and GFP⁻ and GFP⁺Ly6G⁺ cells were sorted on BD FACS Melody (BD Biosciences). GFP⁻ and GFP⁺Ly6G⁺ cells were seeded at 2.000.000 cells/ml in presence of GM-CSF and TES and incubated for further 24 hours. Pellets and supernatants were collected for further analysis. PGE2 concentration in supernatants was measured by using PGE2 ELISA KIT (Invitrogen), according to manufacturer's instructions.

### Uptake and Tracing of Arachidonic Acid

Splenic PMN-MDSC from WT and FATP2 KO LLC TB mice were cultured in complete media (RPMI+10% FBS) with 100 nM of d11AA (Cayman Chemicals), conjugated to 10% fatty acid-free BSA (Sigma Aldrich), and 10 ng/ml of GM-CSF. After 16-18 h, supernatants and cellular pellets were collected and stored at −80c. Lipids (including PGE2 and PGE2d11) were analyzed by LC-MS; the amounts of PGE2 in the supernatants were measured by ELISA.

### Immunofluorescence Microscopy

Immunofluorescence staining of CD8 was performed on frozen mouse tumor tissue sections. Rat monoclonal anti-mouse CD8a primary antibody (BD Pharmingen™) and Alexa Fluor 594 goat anti-rat IgG (H+L) secondary antibody (Invitrogen) were used for the staining. Cell nuclei were stained with DAPI. Imaging was performed using a Leica TCS SP5 confocal microscope. Sixteen frames acquired with a 63× objective lens were used to calculate the cell number per mm².

### Seahorse Analysis

Metabolic rates were determined using the Seahorse XFe24 and XFe96 Flux Analyzers (Agilent Technologies) following the manufacturer's protocol. Briefly, the microplate was coated with 22.4 μg/ml Cell-Tak (Fisher) using 200 mM sodium bicarbonate. 400,000 cells were seeded per well immediately after isolation in 50 μl and 80 μl of unbuffered RPMI (Sigma-Aldrich) for the XF24 and XF96 analyzers, respectively. The microplate was incubated for 30 min at 37° C. to allow the cells to settle into a monolayer. Unbuffered RPMI was gently added to the wells without disturbing the monolayer to bring the assay volume to 675 μl and 1800 for the XFe24 and XFe96 analyzer, respectively. The basal oxygen consumption rate (OCR) and extracellular acidification rate (ECAR) was measured, in addition to rate changes upon treatment with 5 μM oligomycin (Sigma-Aldrich), 1 μM FCCP (Sigma-Aldrich), and 0.75 μM rotenone and 1 μM antimycin A (Sigma-Aldrich).

### Chromatin Immunoprecipitation (ChIP)

Chromatin immunoprecipitation (ChIP) assays were performed as described previously³⁵. Briefly, PBMC cells were treated or not with GM-CSF 10 ng/ml and 100 ng/ml for 20 min. Cells then were fixed in 1% formaldehyde for 10 min. DNAs were sonicated to obtain 200- to 400-bp DNA fragments on a Diagenode Bioruptor according to the manufacturer's protocol. The following antibodies were used for ChIP assays: anti-rabbit IgG (Santa Cruz Biotechnology), anti-Phospho-STAT5 alpha (Tyr694) Antibody (6H5L15) Rabbit Monoclonal (Invitrogen). PCR data were normalized to input values that were quantified in parallel for each experiment.

### Tumor Cell Injections and Treatment

5×10⁵ tumor cells were injected s.c. into mice, which formed tumors with a 1.5-cm diameter within 2-3 weeks of injection. Lipofermata was administrated s.c. at dose of 2 mg/kg twice per day. As a control, mice were treated with vehicle alone (DMSO+30% v/v Kolliphor). Treatments with lipofermata started 8-10 days after tumors injections. CSF1R antibody (BioXcell, 300 μg/mouse) was administered every other day starting next day after tumor injection and continued until the mice were sacrificed. PD-1 antibody (clone RMP1-14, BioXcell, 200 μg/mouse) was administered twice a week started 10-12 days after tumor injection. CTLA4-IgG2a (BioXcell, 200 μg/mouse) was administered at day 7 and day 11.

### Liquid Chromatography-Mass-Spectrometry of Lipids

Lipids were extracted by Folch procedure with slight modifications, under nitrogen atmosphere, at all steps. LC/ESI-MS analysis of lipids was performed on a Dionex HPLC system (utilizing the Chromeleon software), consisting of a Dionex UltiMate 3000 mobile phase pump, equipped with an UltiMate 3000 degassing unit and UltiMate 3000 autosampler (sampler chamber temperature was set at 4° C.). The Dionex HPLC system was coupled to an Orbitrap Fusion Lumos mass spectrometer (ThermoFisher Scientific) or to a hybrid quadrupole-orbitrap mass spectrometer, Q-Exactive (ThermoFisher, Inc., San Jose, Calif.) with the Xcalibur operating system. The instrument was operated in negative and positive ion modes (at a voltage differential of −3.5-5.0 kV, source temperature was maintained at 150° C.). Phospholipids (PLs) MS and MS/MS analysis was performed on an Orbitrap Fusion Lumos mass spectrometer. MS and MS/MS analysis of free fatty acids (FFA) and TAG/CE was performed on a Q-Exactive hybrid-quadrupole-orbitrap mass spectrometer (ThermoFisher, Inc. San Jose, Calif.). MS lipid standards were from Avanti Polar Lipids (Alabaster, Ala.) and from Cayman Chemical Company (Ann Arbor, Mich.). Analysis of LC-MS data was performed using the software package Compound Discoverer™ (ThemoFisher Scientific).

### Construction of LV-GFP-FATP2 Plasmid

The FATP2 gene was amplified from the pCMV6-Kan/Neo-FATP2 plasmid (Origene, Cat #MC206275) using the following primers: FATP2_For_XmaI (GGTGGTCCCGGGCCTATGCTGCCAGTGCTCTACAC) (SEQ ID NO: 6) and FATP2_Rev_SalI (GGTGGTGTCGACTCAGAGCTTCAGAGTTTTAT) (SEQ ID NO: 7). The amplified PCR product was then digested with XmaI/SalI and cloned into a SIV-based self-inactivating lentiviral transfer vector downstream of GFP (pGAE-CMV-GFP-P2A-FATP2-Wpre). The transfer vector pGAE-CMV-GFP-Wpre, the packaging plasmid pAd-SIV3+, and the vesicular stomatitis virus envelope G protein (VSV-G) pseudotyping vector from Indiana serotype (pVSV.GIND), have been previously described^{36,37}.

### Statistical Analysis

Statistical analysis was performed using unpaired two-tailed Student's t-test with significance determined at 0.05. Estimation of variation within each group of data was performed and variance was similar between groups that were compared. Animal experiments were not blinded. Tumor growth was evaluated using two-way Anova test with Bonferroni correction for multiple comparisons.

### Data Availability Statement

The data that support the findings of this study are available from the corresponding author upon reasonable request. RNAseq data are deposited to GEO data repository, accession number GSE126885.

### Example 2: Role of Fatty Acid Transporter 2 (FATP2) in Mediating Immune Suppression in Cancer

FATP2 is Selectively Overexpressed by PMN-MDSC and Controls their Suppressive Activity

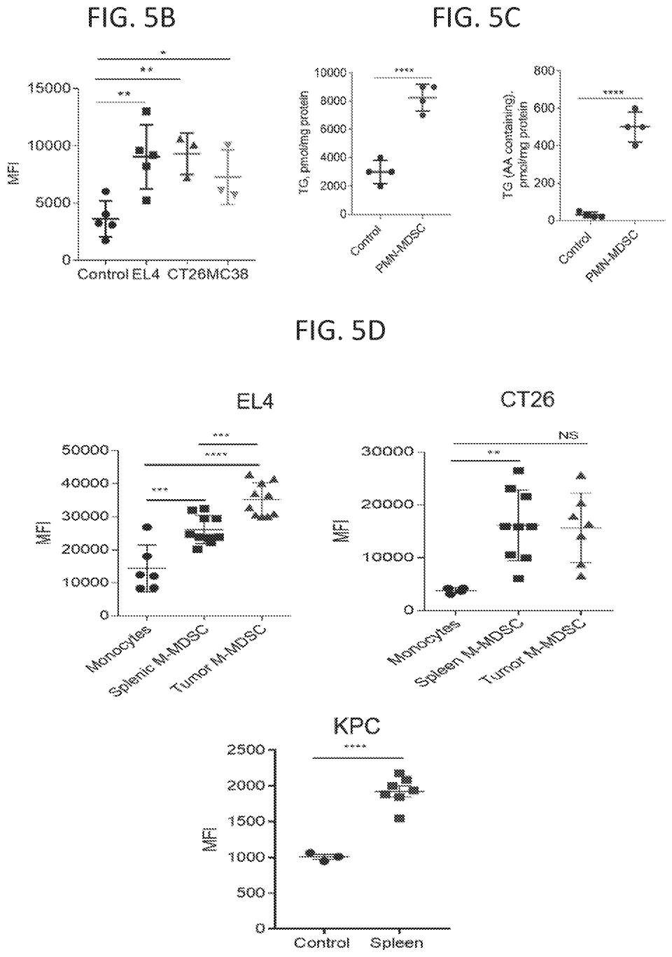

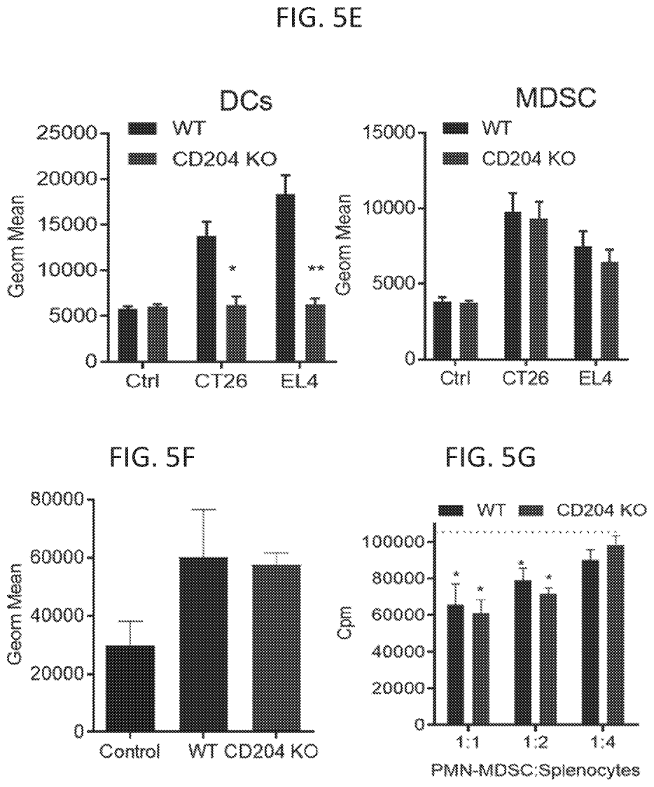

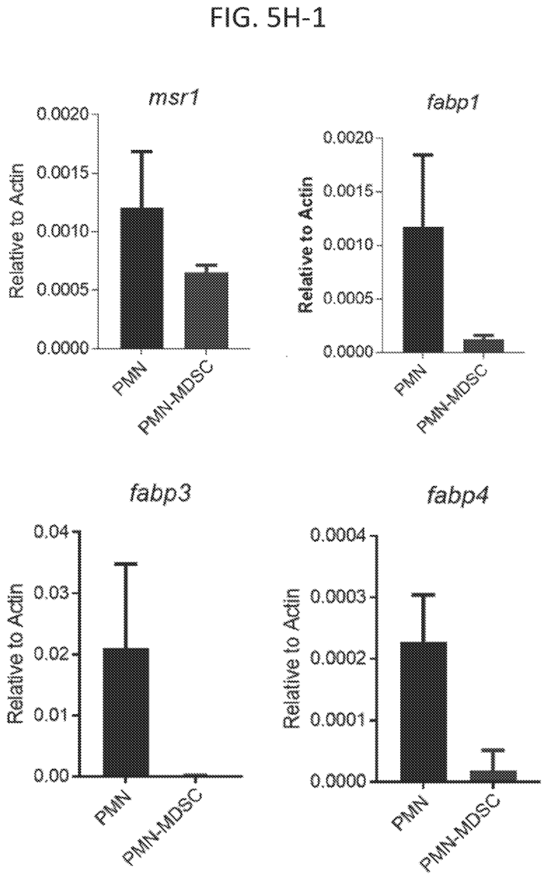

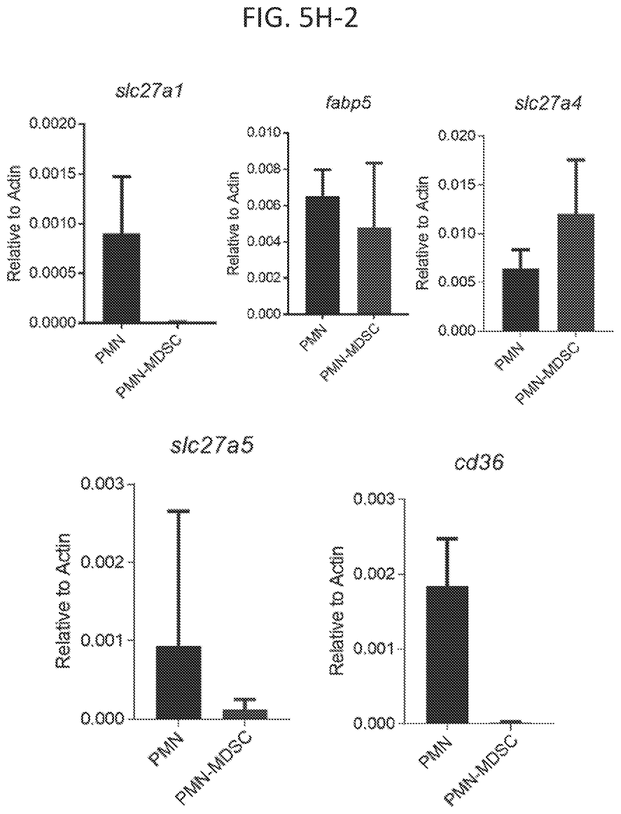

We evaluated total lipid levels in CD11b⁺Ly6C^{lo}Ly6G⁺ PMN-MDSC from spleens of tumor-bearing (TB) mice and neutrophils (PMN) with the same phenotype from spleens of tumor-free mice in transplantable models of EL-4 lymphoma, LLC lung carcinoma, and CT26 colon carcinoma as well as genetically engineered model (GEM) of pancreatic cancer (KPC). PMN-MDSC in all tested models showed substantially higher amounts of lipids than control PMN (FIG. 5A). Tumor explant supernatant (TES) promoted accumulation of lipids in PMN differentiated in vitro from bone marrow (BM) hematopoietic progenitor cells (HPC) (FIG. 5B). LC/MS lipidomics analysis of triglycerides (TG), the major component of lipid droplets¹³ revealed that PMN-MDSC from spleen of TB mice had significantly more TG, than PMN from control mice (FIG. 5C). This effect was particularly robust (˜8-fold) in TG containing arachidonic acid (AA). A similar analysis was performed in CD11b⁺Ly6C^{hi}Ly6G⁻ M-MDSC from TB mice and monocytes with the same phenotype from tumor-free mice. In all tested models, M-MDSC had markedly increased lipid accumulation (FIG. 5D).

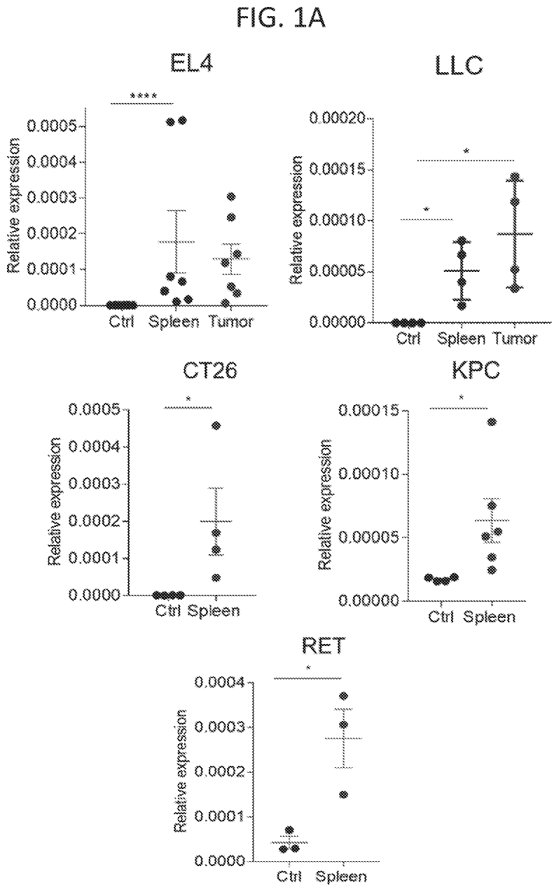

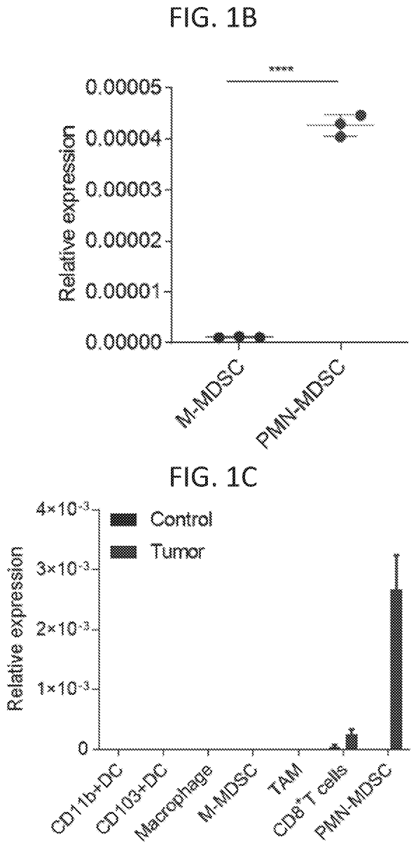

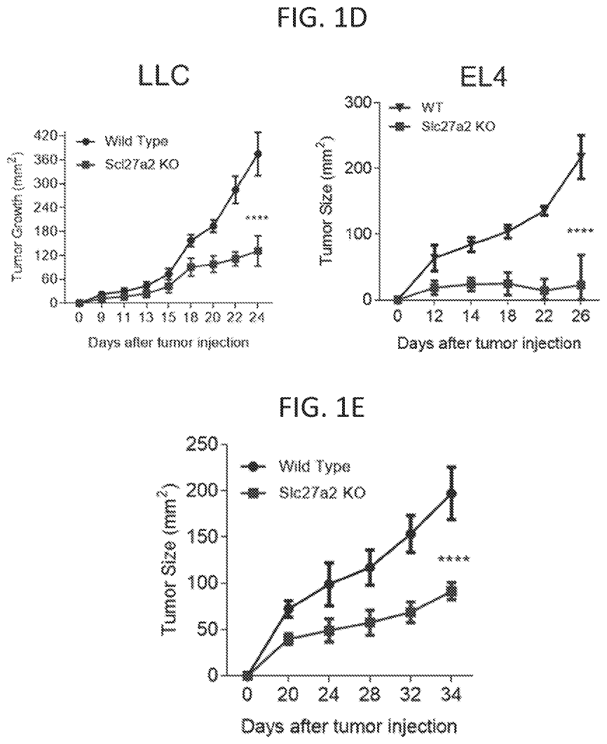

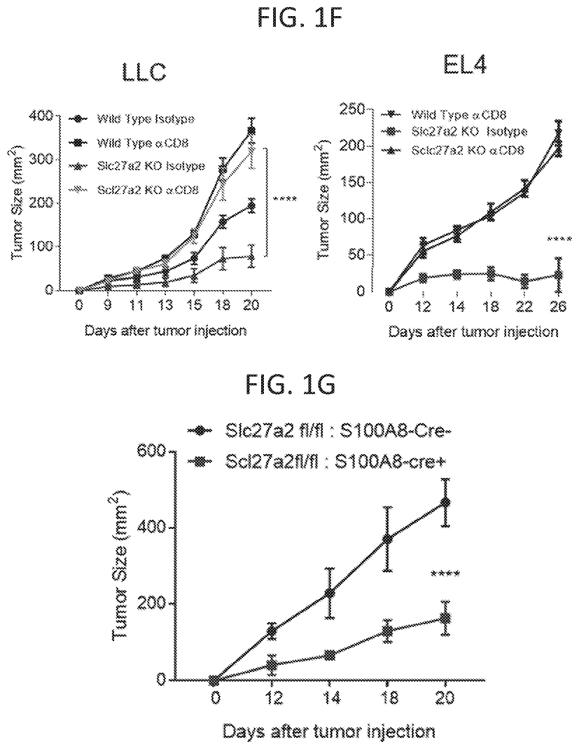

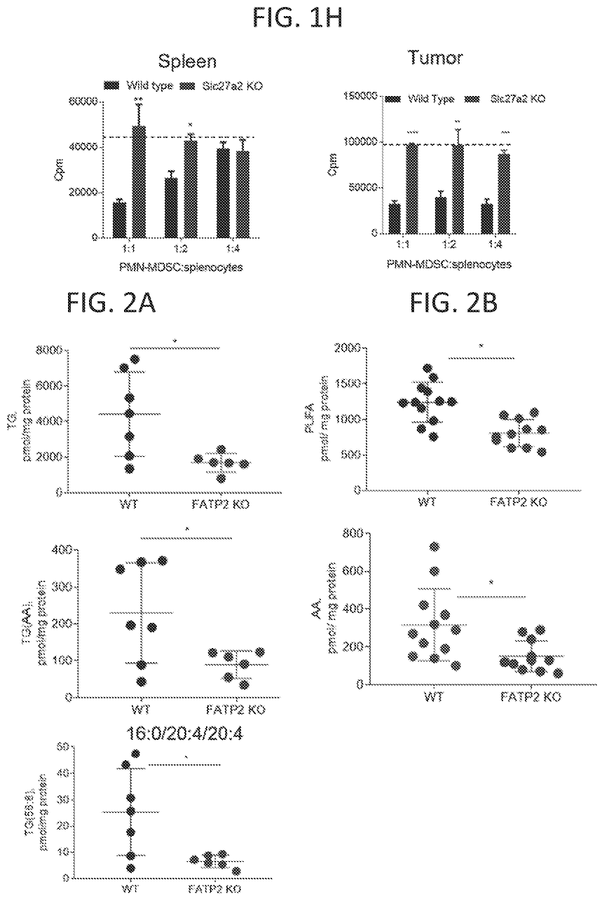

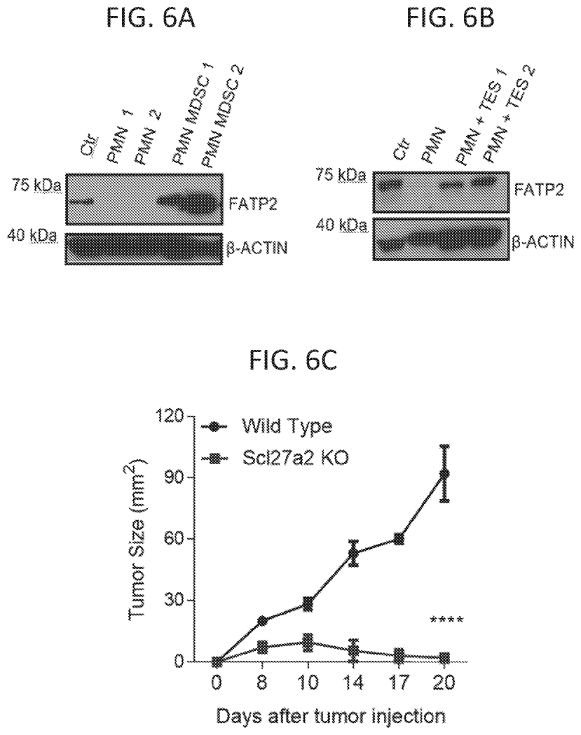

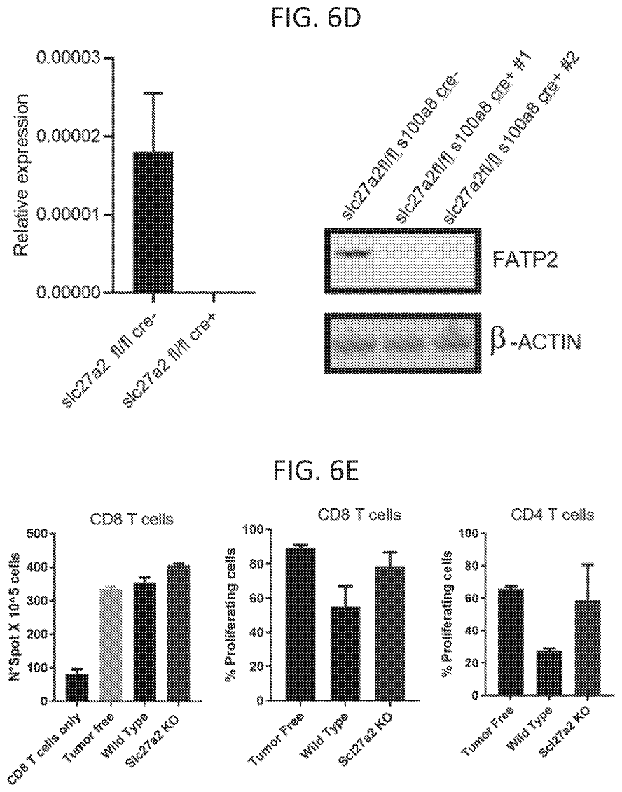

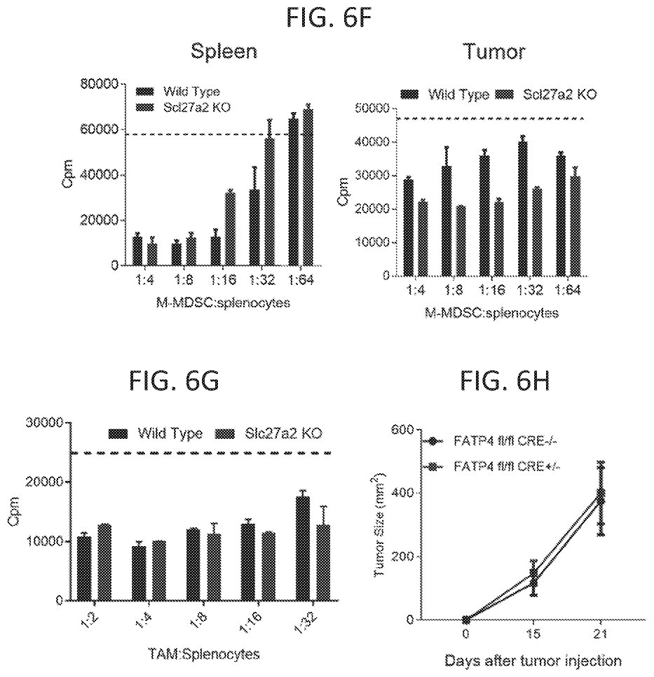

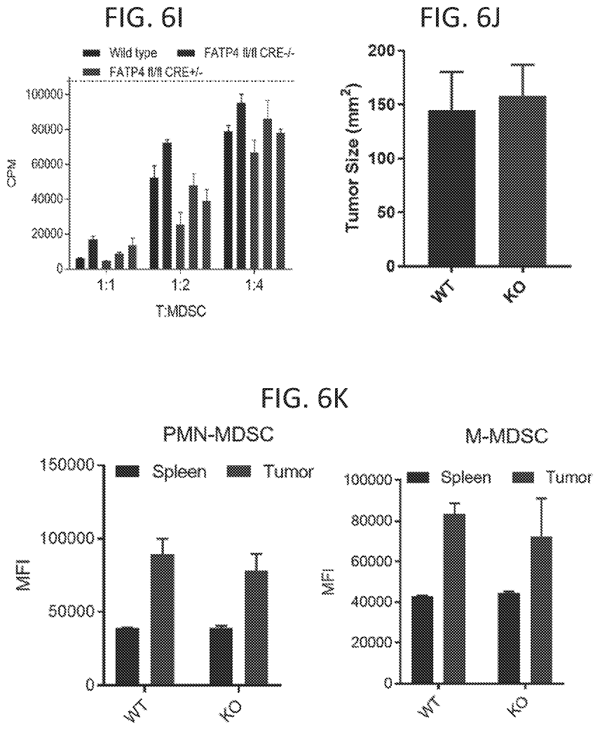

Previous studies demonstrated that lipid accumulation in DCs was mediated by up-regulation of scavenger receptor CD204⁸⁻¹⁰. However, whereas the accumulation of lipids was abrogated in CD204 deficient (Msr1^{-/-}) DCs, it was not affected in PMN (FIG. 5E). These results were confirmed in vivo using BM chimeras of Msr1^{-/-} and wild-type (WT) mice. Lack of CD204 did not abrogate lipid uptake by PMN-MDSC (FIG. 5F) and did not cancel their suppressive activity (FIG. 5G). Several membrane proteins have been implicated in trafficking of lipids, including CD206, CD36, FA binding proteins and FA transport proteins (FATP). The FATP family includes six members (FATP1-6), also known as solute carrier 27 (SLC27). FATP acts as long-chain FA transporter and an acyl-CoA synthetase (ACS)¹⁴⁻¹⁶. ACS converts free long-chain FA into fatty acyl-CoA esters, which can be used in many metabolic processes, including FA synthesis, oxidation, and complex lipid synthesis. We compared the expression of genes potentially involved in lipid uptake between PMN-MDSC from EL4 TB mice and control PMN using the gene expression array described previously¹⁷. PMN-MDSC had a much higher expression of slc27a2, which encodes FATP2. This was confirmed by qPCR (FIG. 1A). No up-regulation of other transporters and receptors involved in lipid accumulation was detected (FIG. 5H). In contrast to PMN-MDSC, M-MDSC showed a barely detectable expression of slc27a2 in the same TB mice (FIG. 1B). DCs, spleen and tumor associated macrophages (TAM) had undetectable and CD8⁺ T cells very low expression of slc27a2 (FIG. 1C). Increased amount of FATP2 protein was confirmed by Western blot in PMN-MDSC isolated from spleens of TB mice (FIG. 6A) or generated in vitro with TES (FIG. 6B).

Next, we asked whether FATP2 might regulate the functionality of PMN-MDSC. To this end, we analyzed the function of PMN-MDSC isolated from slc27a2^{-/-} mice. These mice were originally generated on SV129 background. Therefore, we established a syngeneic sarcoma (F244) in slc27a2^{-/-} and WT mice. Tumors were spontaneously rejected in FATP2 KO mice (FIG. 6C). Slc27a2^{-/-} mice were then backcrossed for 10 generations to C57BL/6 background. We found that in these mice, the growth of LLC and EL4 tumors was markedly slower than in WT mice (FIG. 1D). To test whether that effect was mediated by hematopoietic cells, we established BM chimeras by reconstituting lethally irradiated recipient congenic mice with WT or FATP2 KO BM cells. Tumors established in mice reconstituted with FATP2 KO BM cells grew substantially slower than did tumors in mice reconstituted with WT BM cells (FIG. 1E). Depletion of CD8+ T cells LLC or EL4 TB mice completely abrogated the antitumor activity observed in FATP2 KO mice (FIG. 1F). To confirm a specific role of FATP2 depletion in PMN in the observed antitumor effect, we generated slc27a2^{fl/fl} mice and crossed them with S100A8-cre mice to target the deletion to PMN (FIG. 6D). In the absence of FATP2 in PMN, the tumor grew markedly slower than in control mice (FIG. 1G). Loss of FATP2 did not affect the functionality of CD8⁺ T cells (FIG. 6E).

Since the functionality of PMN-MDSC depends on tumor burden, we compared PMN-MDSC from WT and FATP2 KO TB mice depleted of CD8 T cells, which allow for the analysis of mice with the same tumor size. In both, spleens and tumors of FATP2 KO mice, PMN-MDSC lost the ability to suppress antigen-specific CD8⁺ T cell responses (FIG. 1H). In contrast, the suppressive activity of M-MDSC (FIG. 6F) or TAM (FIG. 6G) was not affected.

Expression of slc27a4, which encodes FATP4 was slightly up-regulated in PMN-MDSC (FIG. 5I). However, in contrast to FATP2 KO mice, no difference in tumor growth and suppressive function of PMN-MDSC were found between WT and FATP4 KO TB mice (FIG. 6H, FIG. 6I). CD36 has been shown to affect the lipid accumulation in different myeloid cells. Since tumors in CD36 KO mice may grow slower than in WT mice and growth depends on CD8 T cells¹², we analyzed the lipid levels in PMN-MDSC from CD36 KO and WT mice with CD8 T cell depletion. We found no difference in lipid accumulation in KO and WT PMN-MDSC (FIG. 6J, FIG. 6K).

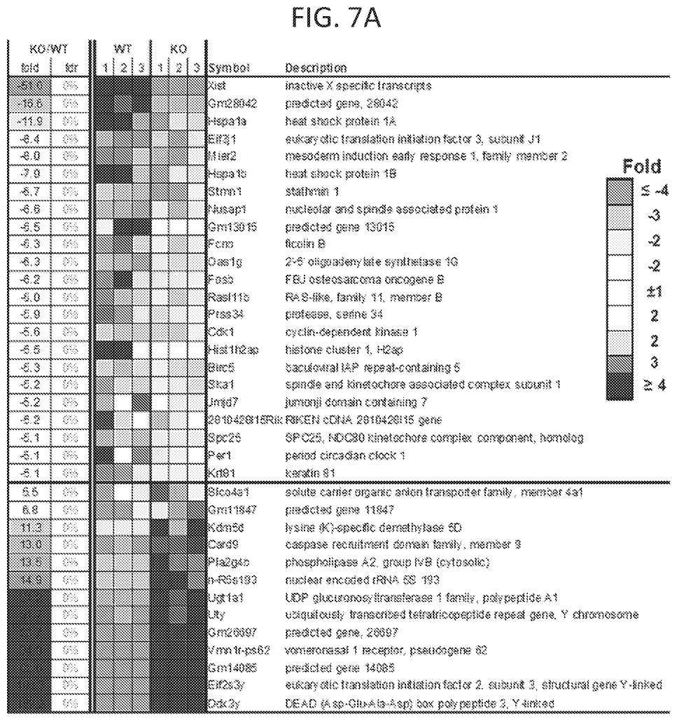

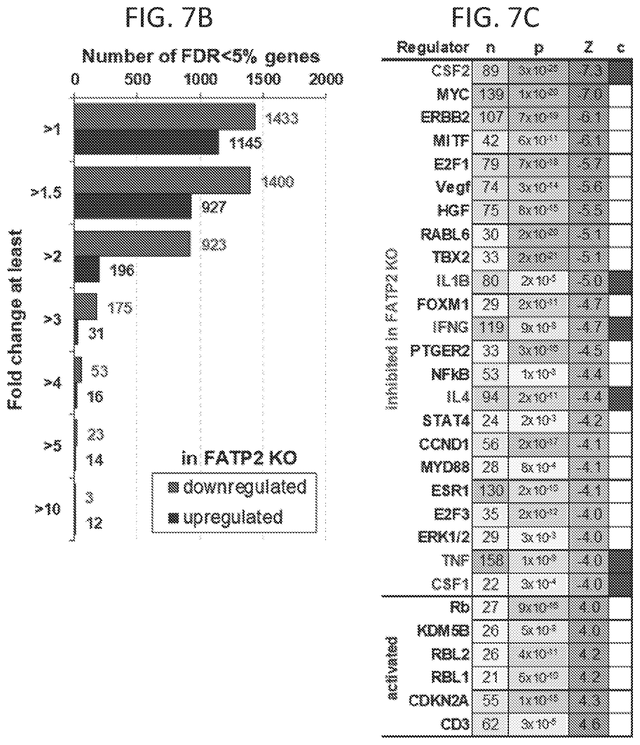

Whole genome RNAseq was performed on spleen PMN-MDSC isolated from WT and FATP2 KO TB mice. Deletion of FATP2 resulted in significant changes in 1119 genes (FRD<5%, at least 2-fold) with 37 genes showing dramatic changes of at least 5-fold (FIG. 7A). There was an overall predominance of genes downregulated in FATP2 KO (FIG. 7B). Enrichment analysis of significantly affected genes using Ingenuity Pathway Analysis revealed that PMN-MDSC from FATP2 KO mice had a marked decrease of pro-inflammatory genes (FIG. 7C).

### FATP2 Regulates Uptake of Arachidonic Acid and PGE2 Synthesis by PMN-MDSC

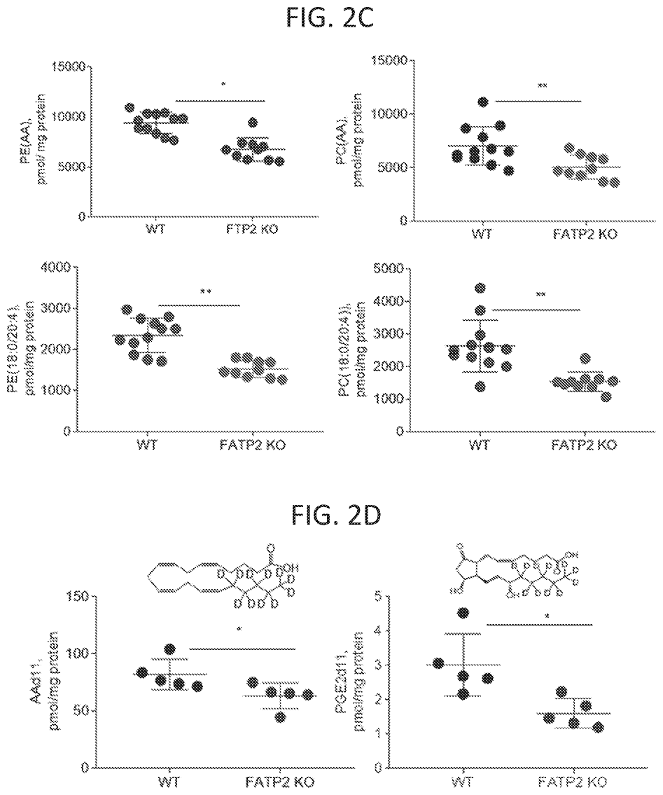

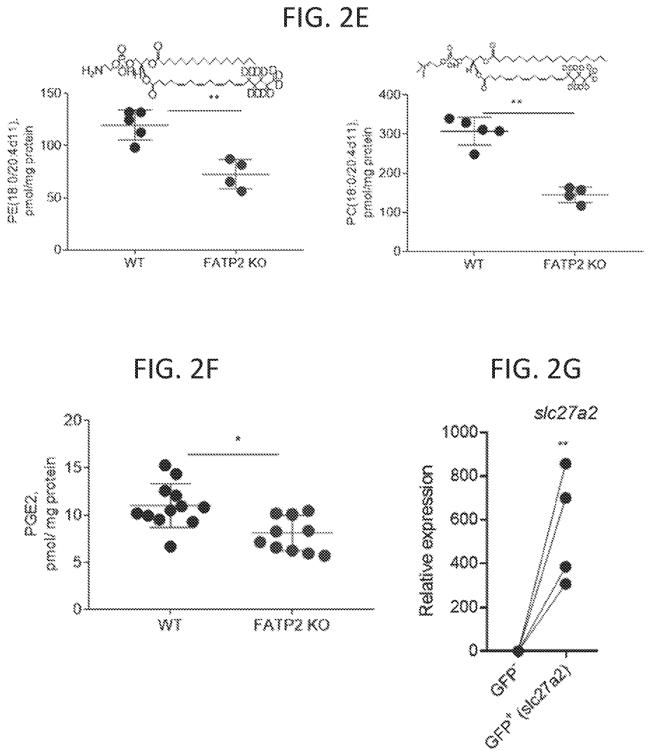

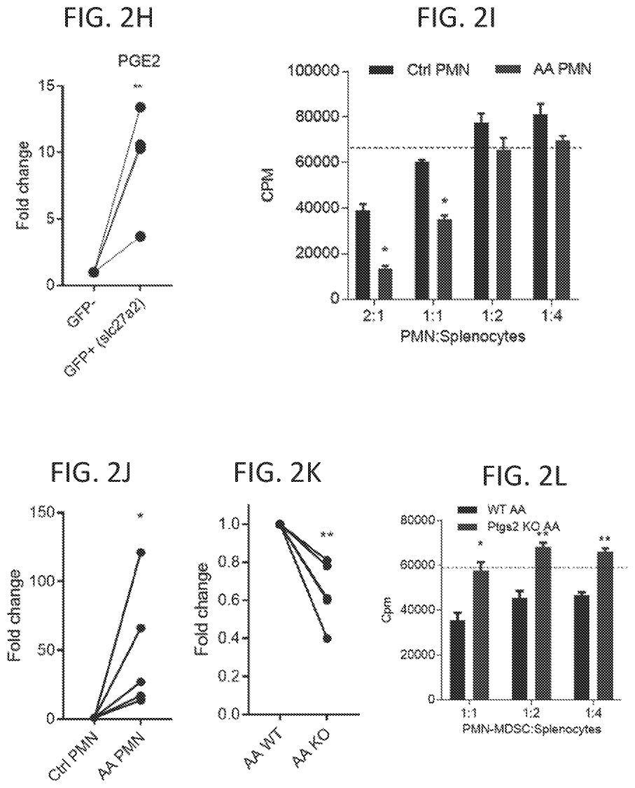

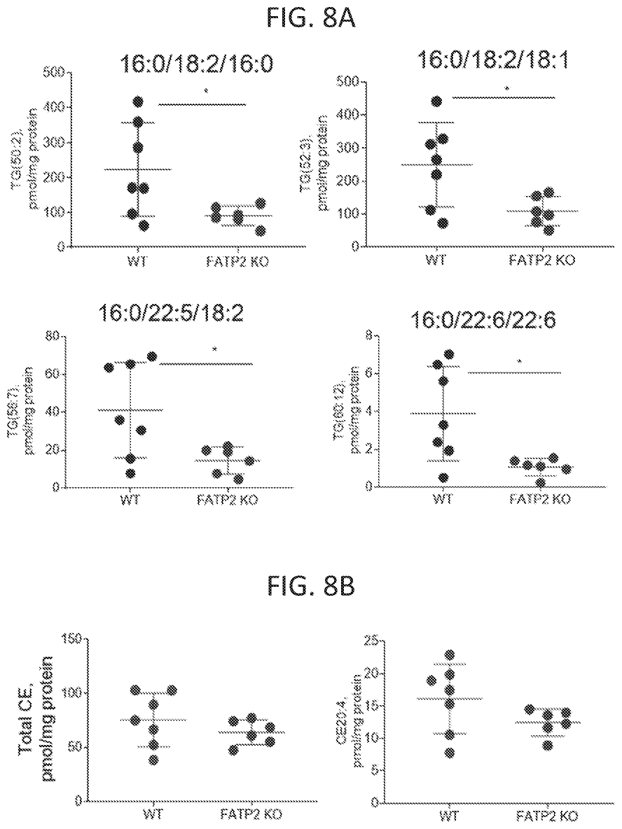

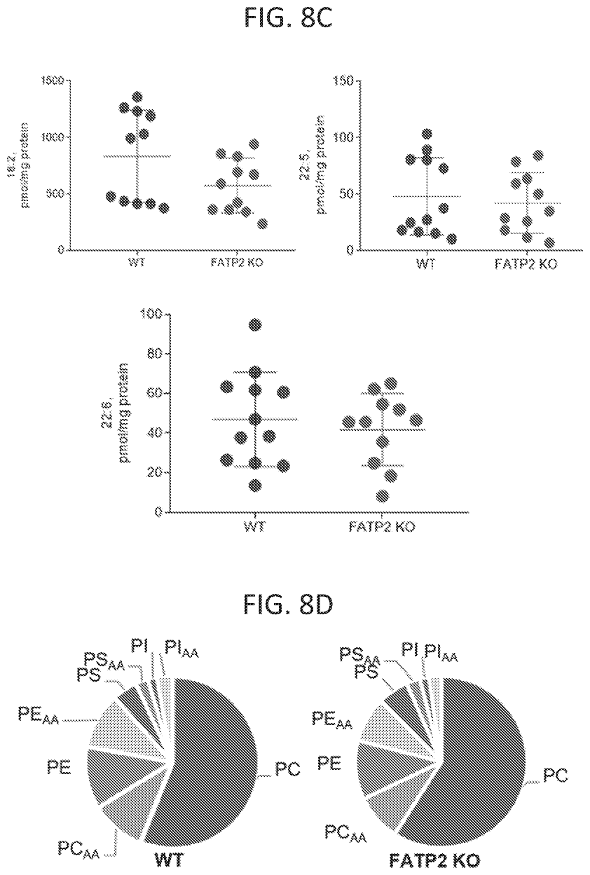

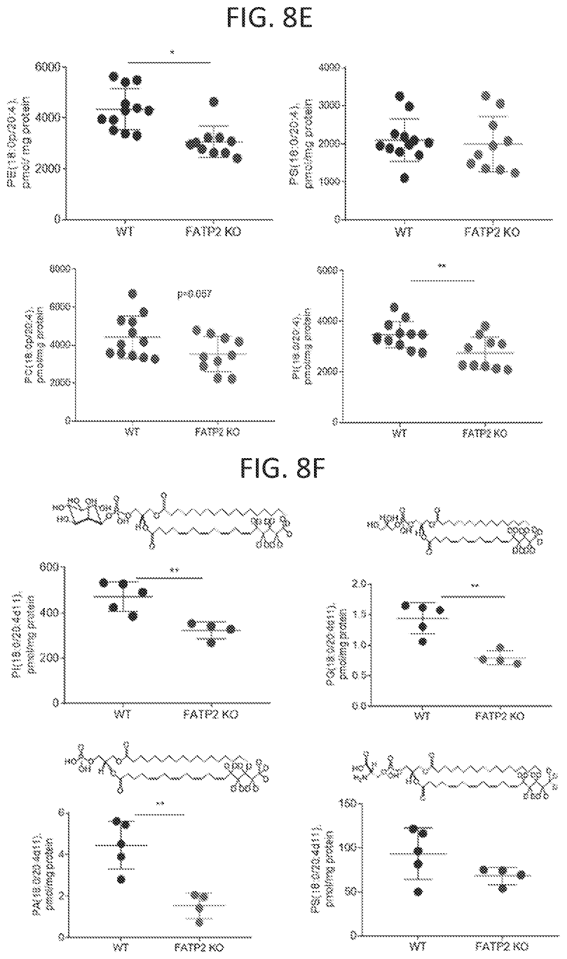

We then investigated the role of FATP2 in regulating lipid accumulation by PMN-MDSC. Experiments were performed with PMN-MDSC isolated from WT and FATP2 KO LLC TB mice with depleted CD8⁺ T cells. LC-MS analysis revealed a lower total amount of TG in FATP2 KO spleen PMN-MDSC than in WT PMN-MDSC and especially TG containing C20:4 AA (FIG. 2A). Polyunsaturated FA (PUFA), AA, C18:2 linoleic acid (LA), C22:5 eicosapentaenoic and C22:6 docosahexaenoic (DHA) FA were markedly reduced (FIG. 2B, FIG. 8A). No differences in the total content of cholesterol esters (CE) or arachidonoyl-containing CE were found (FIG. 8B). The presence of free AA and LA was decreased (FIG. 8C). The total content of phospholipids (PL) was not changed (FIG. 8D), whereas many molecular species of arachidonoyl-containing PL were markedly reduced (FIG. 2C, FIG. 8E). Thus, genetic elimination of FATP2 caused selective depletion of AA-containing species of PL. These finding are consistent with the previous observation that although FATP2 is not a selective transporter for AA, its overexpression favors increased uptake and trafficking of AA16.

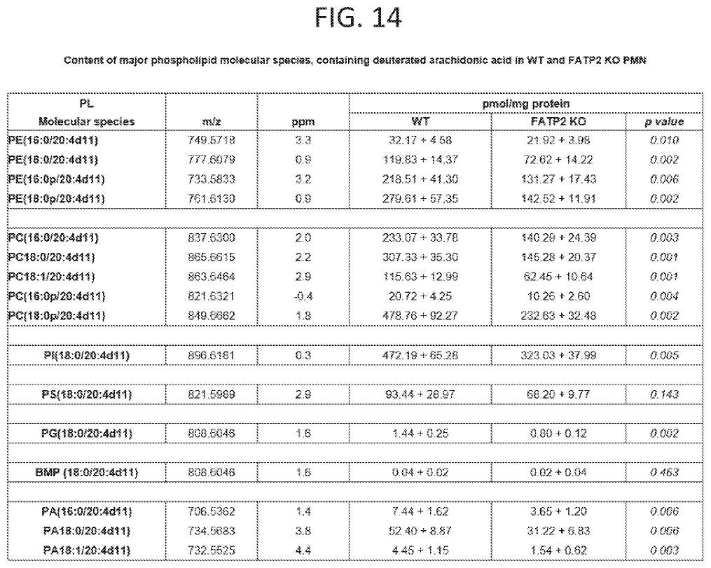

We next employed a method of stable isotope labeling using deuterated AA, (AAd11) and high mass accuracy/high resolution LC-MS with MS/MS fragmentation analysis to directly trace uptake of exogenously added AAd11 by PMN-MDSC from WT and FATP2 KO LLC TB mice. We detected significantly lower amounts of AAd11, as well as labeled PGE2d11 in FATP2 KO PMN-MDSC compared to WT PMN-MDSC (FIG. 2D). We also observed significant reduction of AAd11-containing PL (FIG. 2E, FIG. 14). This was consistent with the markedly reduced amounts of the total (unlabeled) free AA and its metabolite PGE2 (FIG. 8B), as well as unlabeled AA-containing PL (FIG. 8D). No significant differences were observed in the total amounts of palmitic acid (16:0), oleic acid (18:1), LA (18:2), alpha-linolenic acid (18:3), docosapentaenoic acid (22:5), and DHA (22:6) (FIG. 8B).

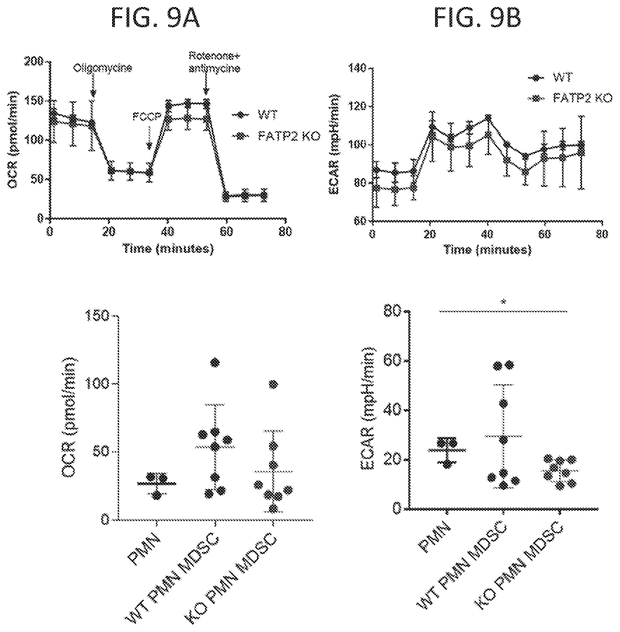

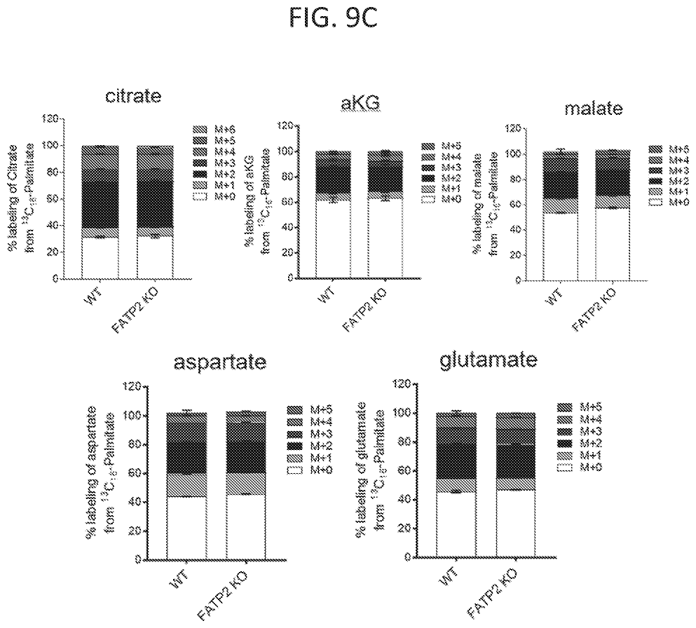

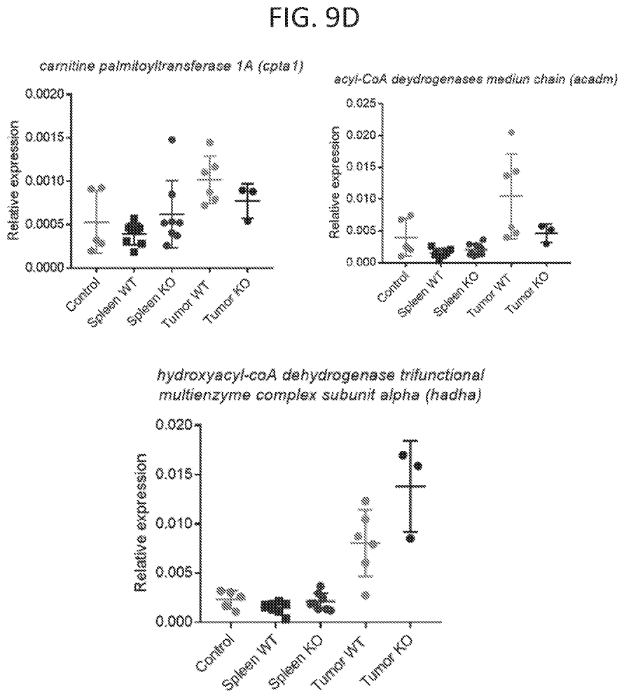

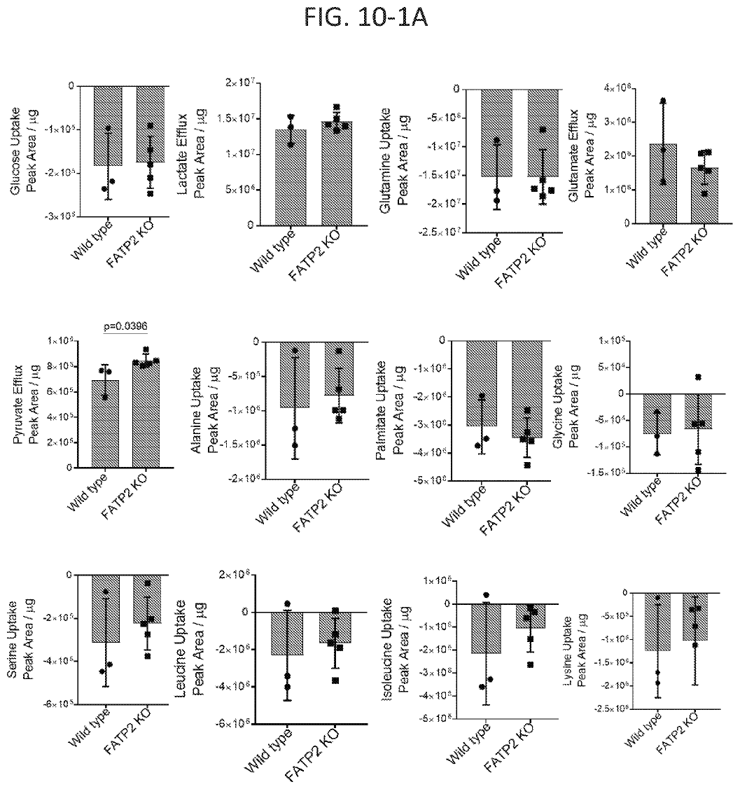

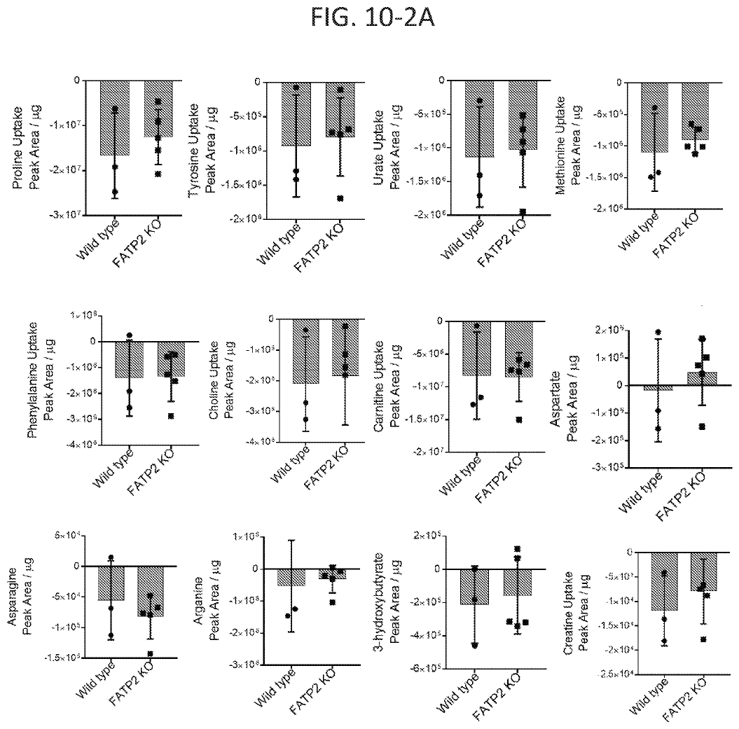

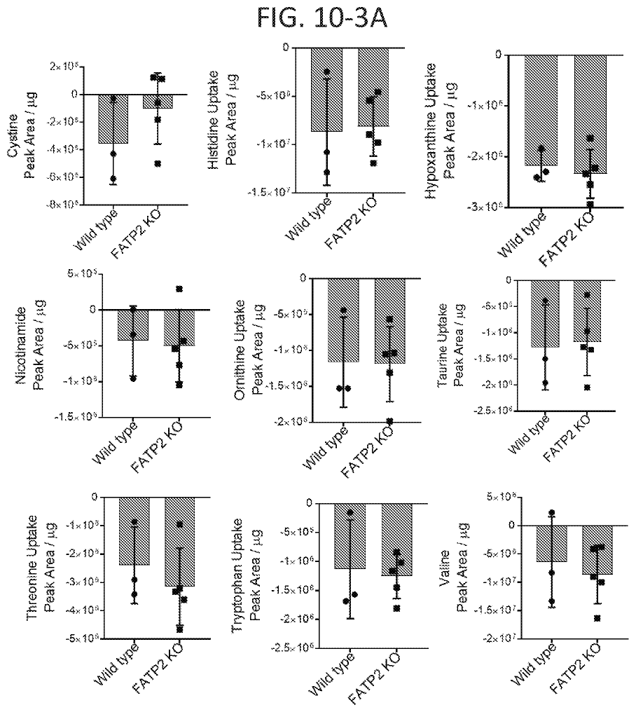

Next, we asked whether lack of FATP2 affected metabolic activity of PMN-MDSC. Spleen PMN-MDSC deficient in FATP2 did not show changes in OXPHOS (FIG. 9A) and glycolysis (FIG. 9B) compared to WT PMN-MDSC. We studied FA oxidation (FAO) in more detail using incorporation of ¹³C₁₆-palmitate to TCA. No differences in labeled metabolites were found between WT and FATP2 KO PMN-MDSC (FIG. 9C). Neither splenic nor tumor PMN-MDSC from FATP2 KO mice showed changes in the expression of cpt1a, hadha, or acadm, major enzymes involved in FAO (FIG. 9D). There were also no differences in the uptake of the major nutrients between WT and FATP2 KO PMN-MDSC (FIG. 10). Taken together, these data indicate that lack of FATP2 does not affect FAO in PMN-MDSC.

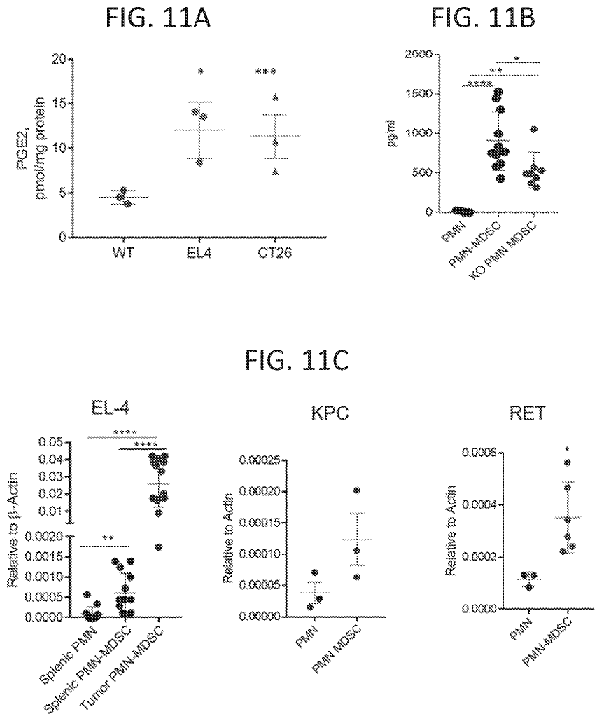

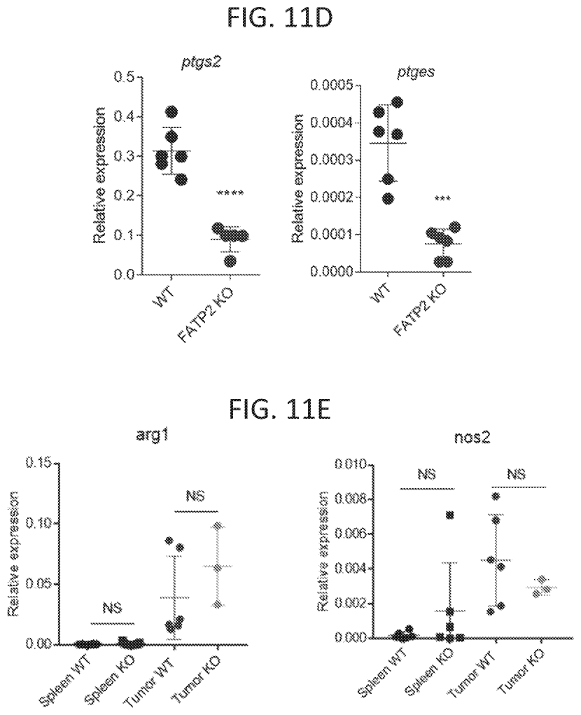

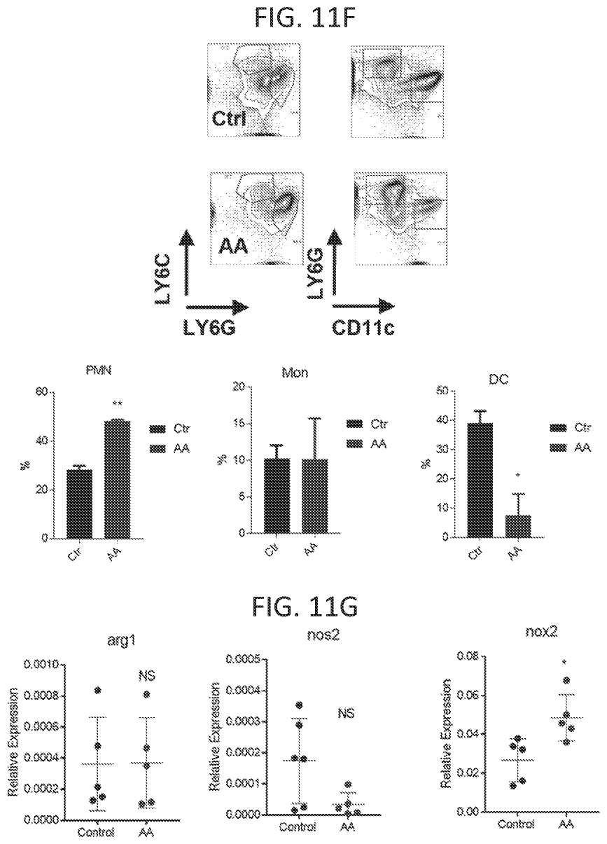

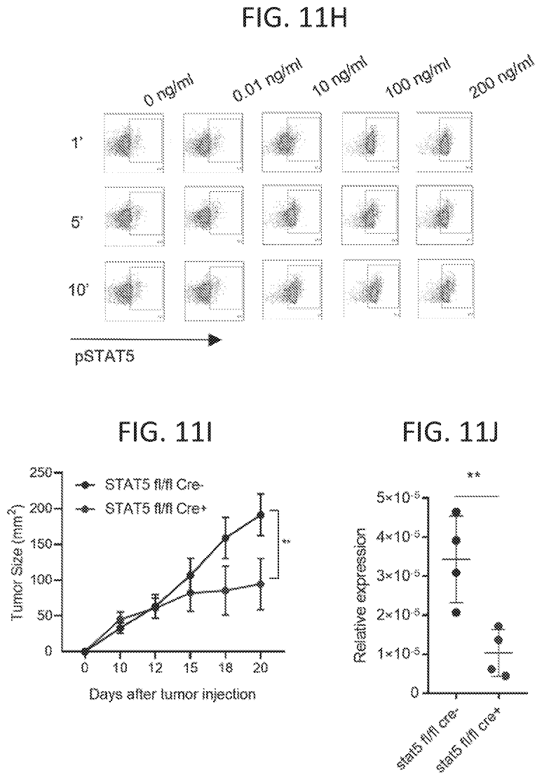

AA is a key precursor of PGE2, which was implicated in the suppressive activity of MDSC in cancer¹⁸⁻²¹ and PMN-MDSC from neonates²². We therefore sought to investigate whether FATP2 regulates the suppressive functions of PMN-MDSC through the accumulation of AA and the subsequent production and release of PGE2. Using LC/MS (FIG. HA) and ELISA (FIG. 11B) we confirmed that PMN-MDSC produced and released significantly higher amount of PGE2 than control PMN. This was associated with higher expression of ptges, prostaglandin E synthase, a key enzyme in the synthesis of PGE2 (FIG. 11C). PMN-MDSC from FATP2 KO TB mice release significantly less PGE2 than WT PMN-MDSC (FIG. 2F). This was consistent with significantly lower amount of intracellular PGE2 in FATP2 deficient PMN-MDSC than in WT cells (FIG. 11B). Consistent with a reduced amount of substrate the expression of genes involved in PGE2 synthesis, ptgs2 and ptges, were lower in FATP2 KO PMN-MDSC than in WT PMN-MDSC (FIG. 11D). No difference was found between WT and KO PMN-MDSC in the expression of genes commonly associated with MDSC activity, arg1, nos2 (FIG. 11E). We transduced HPC with sc127a2-gfp or control lentivirus and differentiated to PMN in the presence of GM-CSF. Overexpression of slc27a2 (FIG. 2G) resulted in increased production of PGE2 in GFP+ PMN as compared to GFP⁻ PMN (FIG. 2H).

To test whether AA could drive the accumulation of suppressive PMN, we generated PMN from HPC in the presence of GM-CSF and AA and found that addition of AA favored the expansion of PMN-MDSC (FIG. 11F) that suppressed antigen specific T cell responses (FIG. 2I). This suppressive activity was associated with a higher production of PGE2 (FIG. 2J), increased expression of nox2, but not arg1 or nos2 (FIG. 11G). To verify the specific role of PGE2 in AA inducible suppressive activity of neutrophils, we generated PMN from COX2 deficient (ptgs2^{-/-}) HPC. In the absence of COX2 PGE2 synthesis was decreased (FIG. 2K). The presence of AA during PMN differentiation from ptgs2^{-/-} HPC was not able to generate suppressive PMN-MDSC (FIG. 2L). Together, these data suggested that FATP2 controls suppressive activity of PMN-MDSC via increased uptake of AA and synthesis of PGE2.

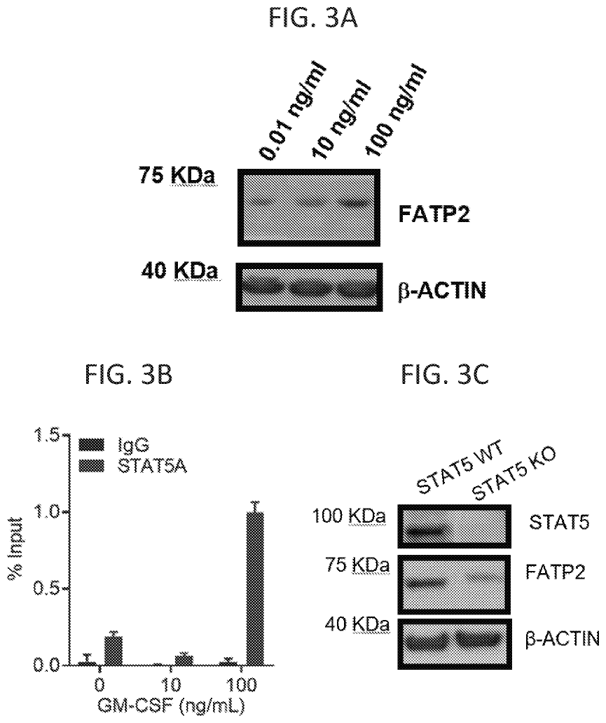

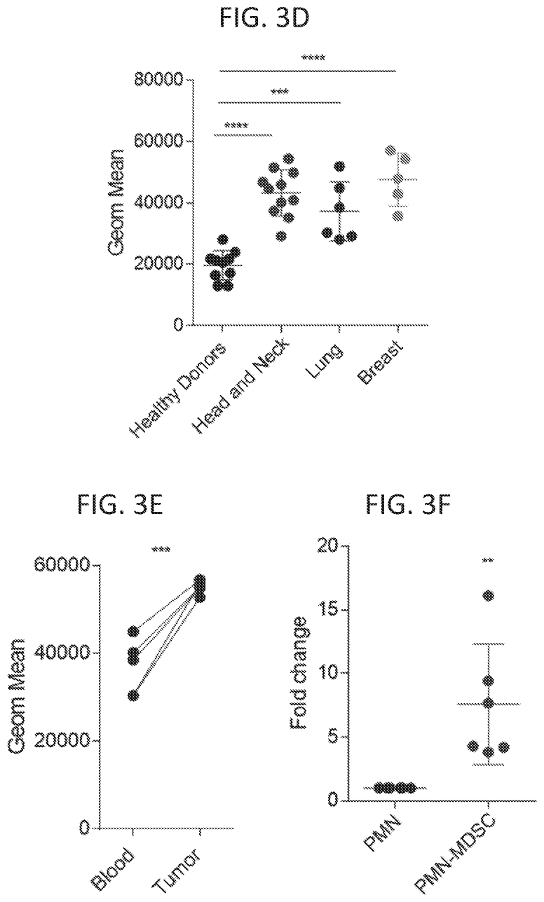

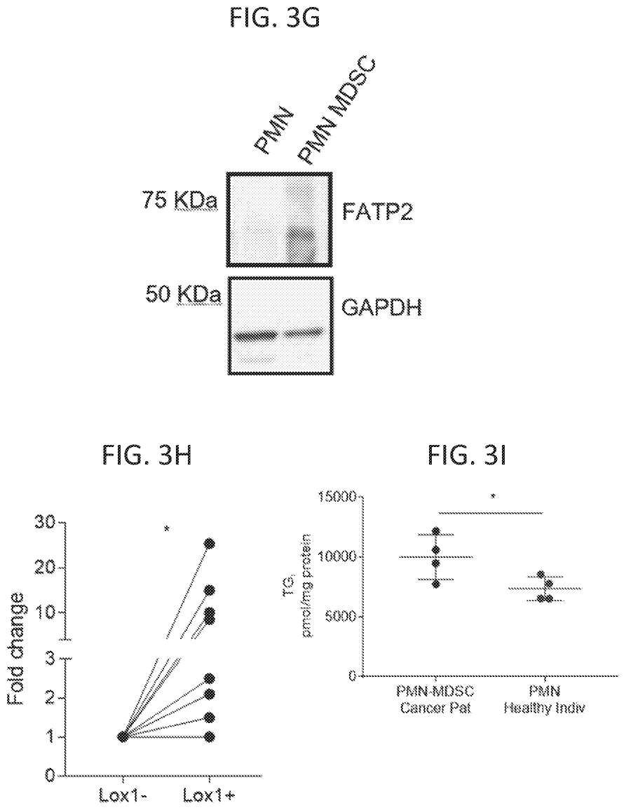

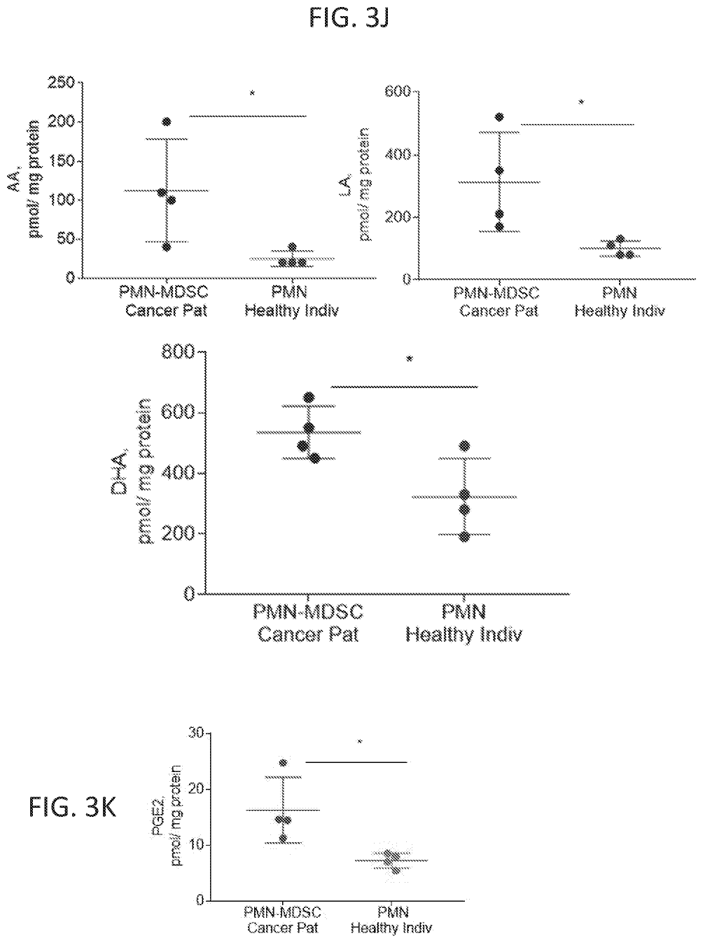

Slc27a2 promoter has a binding site for the transcription factor STAT5 (http://jaspar.genereg.net). STAT5 can be activated by GM-CSF, which plays a critical role in myelopoiesis and expansion of MDSC²³. To explore whether GM-CSF might control slc27a2 expression through STAT5 activation we treated PMN isolated from BM of tumor free mice with GM-CSF for 2 hrs. As expected, it caused a dose-dependent activation of STAT5 (pSTAT5) (FIG. 11H). This activation was associated with up-regulation of FATP2 (FIG. 3A). Chromatin immune precipitation (ChIP) demonstrated that STAT5 could directly bind to the slc27a2 promoter (FIG. 3B). Conversely, GM-CSF failed to increase the expression of FATP2 in Stat5 deficient PMN (FIG. 3C). To confirm the role of STAT5 in controlling the expression of slc27a2 in PMN, we crossed Stat5″ mice with S100A8-cre mice to target the deletion of Stat5 to PMN. In the absence of Stat5 in PMN, the tumor growth was slower than in control mice (FIG. 11I). This was associated with lower expression of slc27a2 in PMN (FIG. 11J). These data indicate that GM-CSF regulates the expression of slc27a2 through the activation of pSTAT5.

### Lipid Accumulation and FATP2 Expression in Human PMN-MDSC

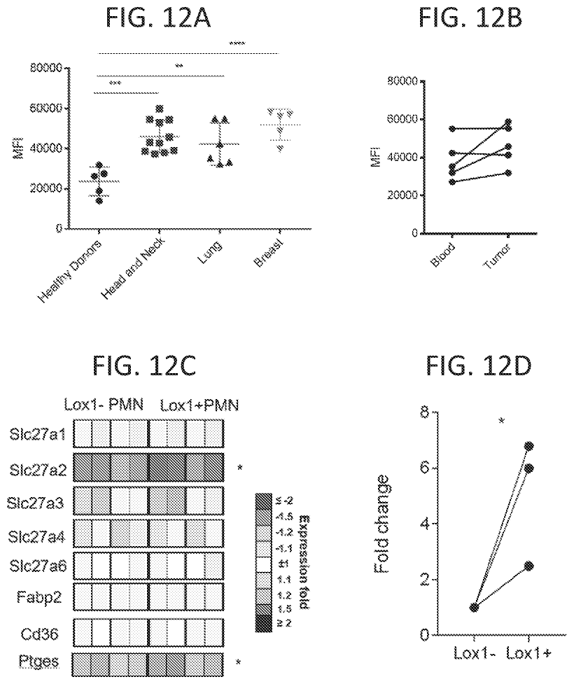

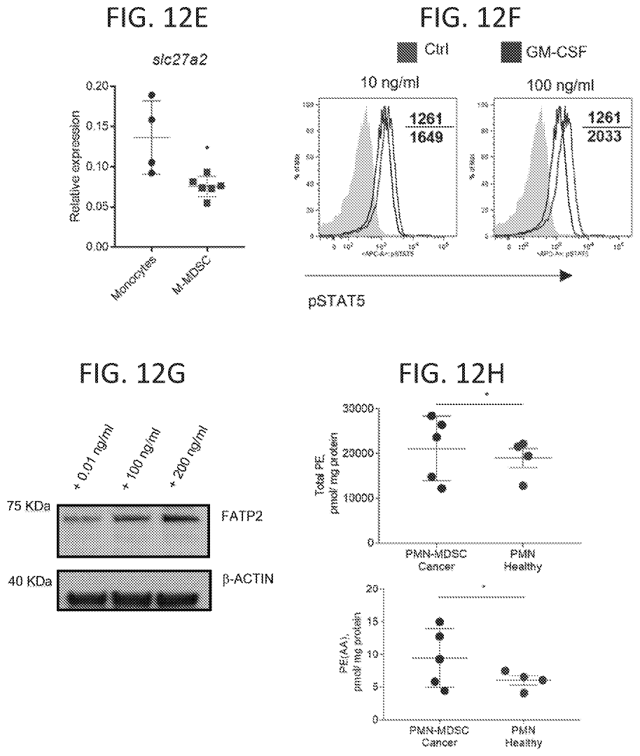

PMN-MDSC isolated from the blood of patients with head and neck, lung, or breast cancers accumulated more lipids than PMN from healthy donors (FIG. 3D). PMN-MDSC in tumors had higher amounts of lipids than PMN-MDSC in blood from the same patients (FIG. 3E). PMN-MDSC from cancer patients had higher expression of SLC27A2 (FIG. 3F) and FATP2 (FIG. 3G) than control PMN. M-MDSC isolated from blood of cancer patients also had more lipids than monocytes from healthy donors (FIG. 12A). However, there was no difference in the accumulation of lipids in M-MDSC isolated from blood and tumor of the same patient (FIG. 12B). Recently, we identified LOX-1 as a marker of human PMN-MDSC²⁴. Analysis of a gene expression array²⁴ revealed that LOX1⁺ PMN-MDSC had higher expression of SLC27A2, but not other transporters as compared with LOX-1⁻ PMN from the same patients (FIG. 12C). The higher expression of SLC27A2 in LOX-1⁺ PMN-MDSC was validated by RT-qPCR (FIG. 3H). SLC27A2 expression was associated with higher expression of PTGES (FIG. 12D). In contrast, M-MDSC had lower expression of SLC27A2 than monocytes (FIG. 12E). Similar to the results obtained in mice, GM-CSF up-regulated pSTAT5 (FIG. 12F), and FATP2 (FIG. 8G).

Using LS/MS lipidomics we identified a substantially higher amount of total TG (FIG. 3I), and free AA, LA, and DHA (FIG. 3J) in PMN-MDSC from cancer patients than in PMN from healthy individuals. Higher amounts of PGE2 were detected in PMN-MDSC than in control PMN (FIG. 3K). The contents of total PE and arachidonoyl-PE (AA-PE) were increased in PMN-MDSC from cancer patients compared with PMN from healthy donors (FIG. 12H). Thus, clinical data recapitulated the observations in mice.

### Therapeutic Targeting of FATP2

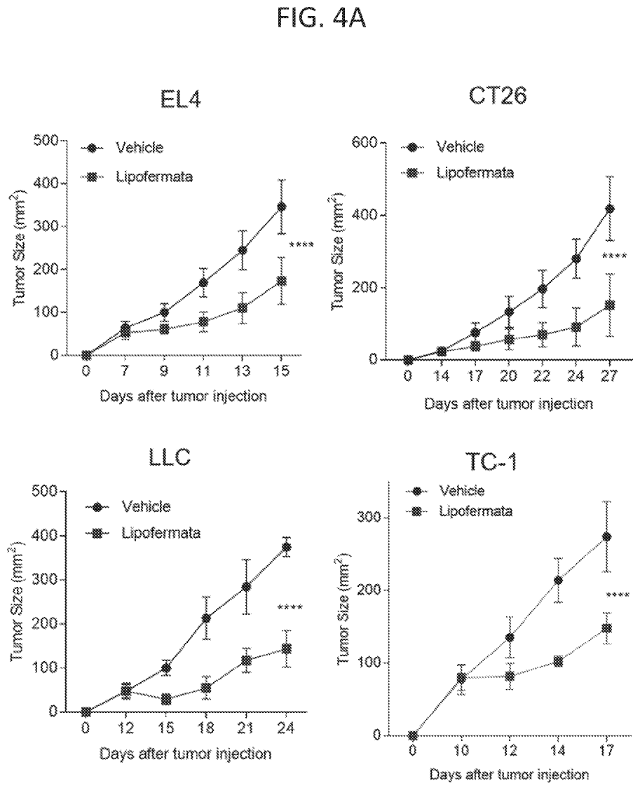

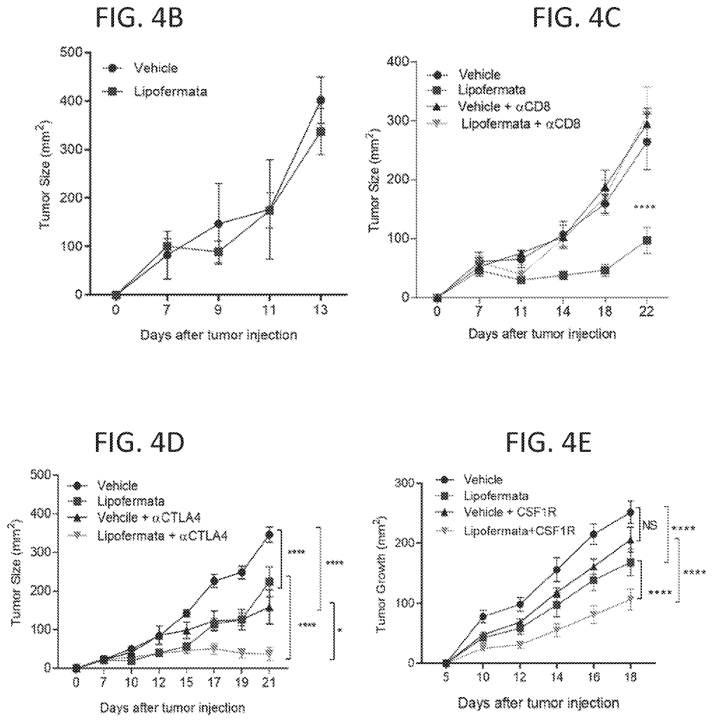

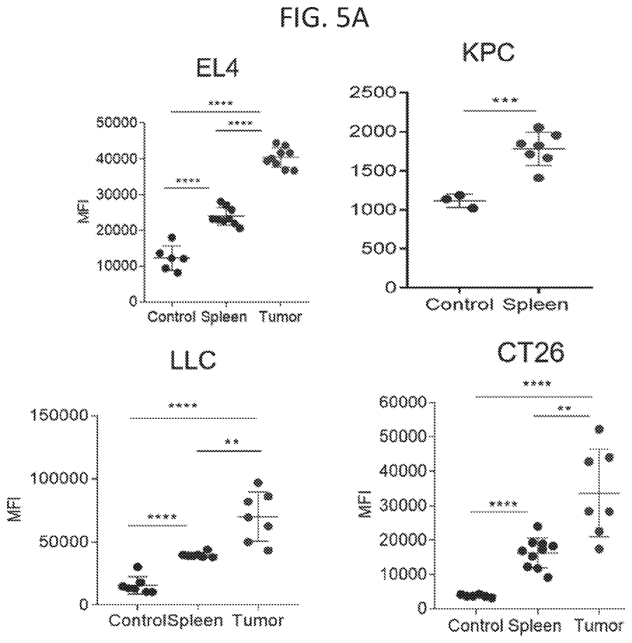

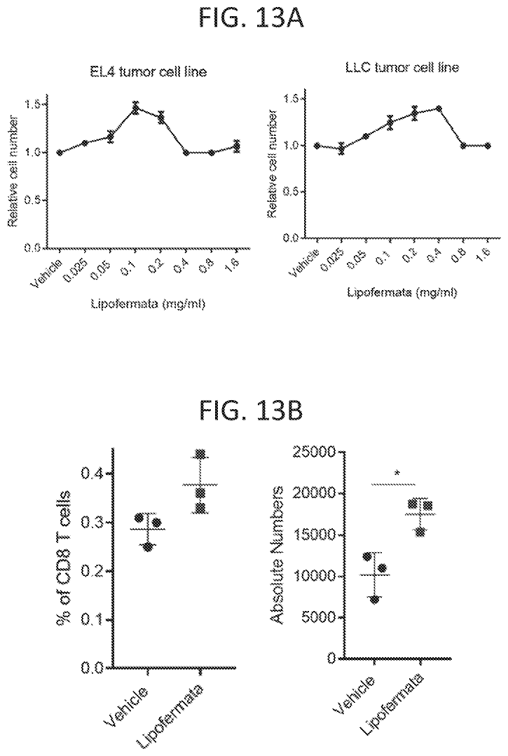

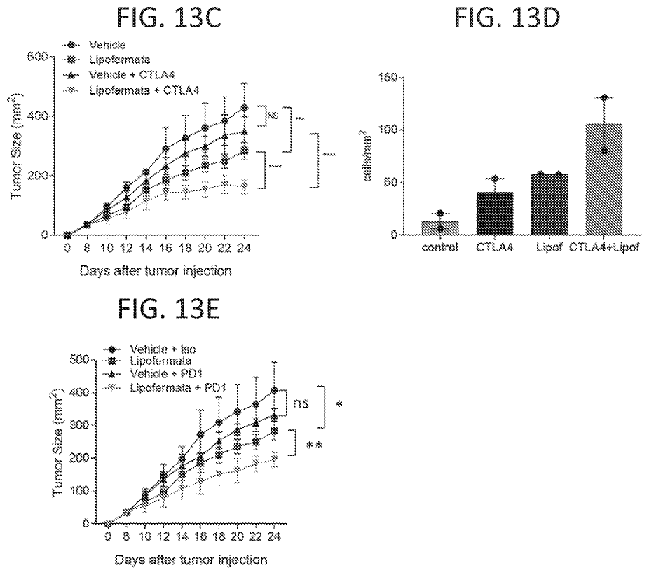

Next, we sought to determine the impact of the pharmacological inhibition of FATP2 on tumor growth. To inhibit FATP2 in TB mice, we used 5′-bromo-5-phenyl-spiro[3H-1,3,4-thiadiazole-2,3′-indoline]-2′-one, (lipofermata). This is a selective FATP2 inhibitor^{15,25}. Lipofermata at the range of concentrations corresponding to the dose used in vivo (0.2 mg/ml) did not affect proliferation of EL-4 and LLC tumor cells in vitro (FIG. 13A). In four tested tumor models, lipofermata caused a significant delay of tumor growth (FIG. 4A). Notably this effect was absent in immune deficient SCID-NOD mice (FIG. 4B), and depletion of CD8⁺ T cells in immune competent mice abrogated the effect of lipofermata (FIG. 4C). These data indicate that antitumor effect of FATP2 inhibition was mediated via immune mechanisms. In the TC-1 model, treatment with lipofermata increased the percentage and absolute numbers of antigen specific T cells in draining lymph nodes (FIG. 13B).

We asked whether lipofermata could provide additional therapeutic benefit if combined with checkpoint inhibitors. Treatment of LLC bearing mice with lipofermata or CTLA4 alone had an antitumor effect. However, neither blocked tumor progression. In contrast, combination of CTLA4 antibody with lipofermata caused potent antitumor effect with 4 out of 5 mice rejecting tumors (FIG. 4D). A similar combination effect was observed in the TC1 model (FIG. 13C). The antitumor effect was associated with substantial infiltration of CD8⁺ T cells of tumors (FIG. 13D). Combination of PD1 antibody with lipofermata in TC-1 model also resulted in significant decrease in tumor growth although this effect was less pronounced (FIG. 13E). Since FATP2 is overexpressed only on PMN-MDSC, we asked whether the antitumor effects of lipofermata could be potentiated by combining with TAM targeted CSF1R antibody. Consistent with previous observations²⁶ CSF1R antibody alone had only a minor effect on tumor growth in the LLC tumor model. Combination of lipofermata with CSF1R antibody resulted in cellular antitumor effect (FIG. 4E).

Our study has identified FATP2 as a critical regulator of the immune suppressive function of PMN-MDSC, which mediates its effect via regulation of the accumulation of AA and subsequent synthesis of PGE2. These findings are consistent with the results demonstrating that production of PGE2 support tumor growth and immune escape²⁷. Previous reports established the potential role of COX2 inhibitors in blockade of MDSC expansion in mouse tumor models^{28,18,29,30.} However, prolonged systemic use of COX2 inhibitors is associated with substantial hematologic, cardiovascular and gastrointestinal toxicities. Selective targeting of FATP2 in PMN-MDSC offers the opportunity to inhibit PGE2 only in pathologically activated neutrophils and mostly within the tumor site, where expression of FATP2 is the highest. It is also possible that blockade of local release of PGE2 at the contact between PMN-MDSC and T cells in peripheral lymphoid organs can improve immune responses without systemic effects of PGE2 inhibition.

### REFERENCES

1. Zhou, J., Nefedova, Y., Lei, A. & Gabrilovich, D. Neutrophils and PMN-MDSC: Their biological role and interaction with stromal cells. Semin Immunol 35, 19-28, doi:10.1016/j.smim.2017.12.004 (2018).
2. Veglia, F., Perego, M. & Gabrilovich, D. Myeloid-derived suppressor cells coming of age. Nat Immunol 19, 108-119, doi:10.1038/s41590-017-0022-x (2018).
3. Gabrilovich, D. I., Ostrand-Rosenberg, S. & Bronte, V. Coordinated regulation of myeloid cells by tumors. Nat Rev Immunol 12, 253-268 (2012).
4. Kumar, V., Patel, S., Tcyganov, E. & Gabrilovich, D. I. The Nature of Myeloid-Derived Suppressor Cells in the Tumor Microenvironment. Trends Immunol 37, 208-220, doi:10.1016/j.it.2016.01.004 (2016).
5. Moore, K. J., Sheedy, F. J. & Fisher, E. A. Macrophages in atherosclerosis: a dynamic balance. Nat Rev Immunol 13, 709-721, doi:10.1038/nri3520 (2013).
6. O'Neill, L. A. & Pearce, E. J. Immunometabolism governs dendritic cell and macrophage function. J Exp Med 213, 15-23, doi:10.1084/jem.20151570 (2016).
7. Hubler, M. J. & Kennedy, A. J. Role of lipids in the metabolism and activation of immune cells. J Nutr Biochem 34, 1-7, doi:10.1016/j.jnutbio.2015.11.002 (2016).
8. Veglia, F. et al. Lipid bodies containing oxidatively truncated lipids block antigen cross-presentation by dendritic cells in cancer. Nature communications 8, 2122, doi:10.1038/s41467-017-02186-9 (2017).
9. Ramakrishnan, R. et al. Oxidized lipids block antigen cross-presentation by dendritic cells in cancer. J Immunol 192, 2920-2931, doi:10.4049/jimmunol.1302801 (2014).
10. Herber, D. L. et al. Lipid accumulation and dendritic cell dysfunction in cancer. Nat Med 16, 880-886 (2010).
11. Cubillos-Ruiz, J. R. et al. E R Stress Sensor XBP1 Controls Anti-tumor Immunity by Disrupting Dendritic Cell Homeostasis. Cell 161, 1527-1538, doi:10.1016/j.cell.2015.05.025 (2015).
12. Al-Khami, A. A. et al. Exogenous lipid uptake induces metabolic and functional reprogramming of tumor-associated myeloid-derived suppressor cells.

Oncoimmunology 6, e1344804, doi:10.1080/2162402X.2017.1344804 (2017).
13. den Brok, M. H., Raaijmakers, T. K., Collado-Camps, E. & Adema, G. J. Lipid Droplets as Immune Modulators in Myeloid Cells. Trends Immunol 39, 380-392, doi:10.1016/j.it.2018.01.012 (2018).
14. Black, P. N. & DiRusso, C. C. Yeast acyl-CoA synthetases at the crossroads of fatty acid metabolism and regulation. Biochim Biophys Acta 1771, 286-298, doi:10.1016/j.bbalip.2006.05.003 (2007).
15. Black, P. N., Ahowesso, C., Montefusco, D., Saini, N. & DiRusso, C. C. Fatty Acid Transport Proteins: Targeting FATP2 as a Gatekeeper Involved in the Transport of Exogenous Fatty Acids. MedChemComm 7, 612-622, doi:10.1039/C6MD00043F (2016).
16. Nicks, K. M. et al. Three-dimensional structural analysis of the proximal femur in an age-stratified sample of women. Bone 55, 179-188, doi:10.1016/j.bone.2013.02.009 (2013).
17. Youn, J. I., Collazo, M., Shalova, I. N., Biswas, S. K. & Gabrilovich, D. I. Characterization of the nature of granulocytic myeloid-derived suppressor cells in tumor-bearing mice. J Leukoc Biol 91, 167-181, doi:10.1189/jlb.0311177 (2012).
18. Rodriguez, P. C. et al. Arginase I in myeloid suppressor cells is induced by COX-2 in lung carcinoma. J Exp Med 202, 931-939 (2005).
19. Sinha, P., Clements, V. K., Fulton, A. M. & Ostrand-Rosenberg, S. Prostaglandin E2 promotes tumor progression by inducing myeloid-derived suppressor cells. Cancer Res 67, 4507-4513 (2007).
20. Li, Y. et al. Myeloid-derived suppressor cells as a potential therapy for experimental autoimmune myasthenia gravis. J Immunol 193, 2127-2134, doi:10.4049/jimmunol.1400857 (2014).
21. Mei, Z., Korotkova, O. & Mao, Y. Products of Schell-model cross-spectral densities. Opt Lett 39, 6879-6882, doi:10.1364/OL.39.006879 (2014).
22. He, Y.-M. et al. Transitory presence of myeloid-derived suppressor cells in neonates is critical for control of inflammation Nat Med in press (2018).
23. Casbon, A. J. et al. Invasive breast cancer reprograms early myeloid differentiation in the bone marrow to generate immunosuppressive neutrophils. Proc Natl Acad Sci USA 112, E566-575, doi:10.1073/pnas.1424927112 (2015).
24. Condamine, T. et al. Lectin-type oxidized LDL receptor-1 distinguishes population of human polymorphonuclear myeloid-derived suppressor cells in cancer patients. Sci Immunol 1, doi:10.1126/sciimmunol.aaf8943 (2016).
25. Ahowesso, C. et al. Chemical inhibition of fatty acid absorption and cellular uptake limits lipotoxic cell death. Biochem Pharmacol 98, 167-181, doi:10.1016/j.bcp.2015.09.004 (2015).
26. Kumar, V. et al. Cancer-Associated Fibroblasts Neutralize the Anti-tumor Effect of CSF1 Receptor Blockade by Inducing PMN-MDSC Infiltration of Tumors. Cancer Cell 32, 654-668 e655, doi:10.1016/j.ccell.2017.10.005 (2017).
27. Zelenay, S. et al. Cyclooxygenase-Dependent Tumor Growth through Evasion of Immunity. Cell 162, 1257-1270, doi:10.1016/j.cell.2015.08.015 (2015).
28. Fujita, M. et al. COX-2 blockade suppresses gliomagenesis by inhibiting myeloid-derived suppressor cells. Cancer Res 71, 2664-2674, doi:10.1158/0008-5472.CAN-10-3055 (2011).
29. Veltman, J. D. et al. COX-2 inhibition improves immunotherapy and is associated with decreased numbers of myeloid-derived suppressor cells in mesothelioma. Celecoxib influences MDSC function. BMC cancer 10, 464, doi:10.1186/1471-2407-10-464 (2010).
30. He, Y. M. et al. Transitory presence of myeloid-derived suppressor cells in neonates is critical for control of inflammation. Nat Med 24, 224-231, doi:10.1038/nm.4467 (2018).
31. Henao-Mejia, J. et al. Generation of Genetically Modified Mice Using the CRISPR-Cas9 Genome-Editing System. Cold Spring Harb Protoc 2016, pdb prot090704, doi:10.1101/pdb.prot090704 (2016).
32. Langmead, B. & Salzberg, S. L. Fast gapped-read alignment with Bowtie 2. Nat Methods 9, 357-359, doi:10.1038/nmeth.1923 (2012).
33. Li, B. & Dewey, C. N. RSEM: accurate transcript quantification from RNA-Seq data with or without a reference genome. BMC bioinformatics 12, 323, doi:10.1186/1471-2105-12-323 (2011).
34. Love, M. I., Huber, W. & Anders, S. Moderated estimation of fold change and dispersion for RNA-seq data with DESeq2. Genome biology 15, 550, doi:10.1186/s13059-014-0550-8 (2014).
35. Chen, H. S. et al. BET-Inhibitors Disrupt Rad21-Dependent Conformational Control of KSHV Latency. PLoS Pathog 13, e1006100, doi:10.1371/journal.ppat.1006100 (2017).
36. Michelini, Z. et al. Development and use of SIV-based Integrase defective lentiviral vector for immunization. Vaccine 27, 4622-4629, doi:10.1016/j.vaccine.2009.05.070 (2009).
37. Negri, D. et al. Immunization with an SIV-based IDLV Expressing HIV-1 Env 1086 Clade C Elicits Durable Humoral and Cellular Responses in Rhesus Macaques. Mol Ther, doi:10.1038/mt.2016.123 (2016).

### Sequence Listing Free Text

The following information is provided for sequences containing free text under numeric identifier <223>.

| SEQ ID NO | Free Text under <223> |
|---|---|
| 2 | <223> engineered construct |
| 3 | <223> engineered construct |
| 4 | <223> engineered construct |
| 5 | <223> engineered construct |
| 6 | <223> primer sequence |
| 7 | <223> primer sequence |

Each and every patent, patent application, and any document listed herein, and the sequence of any publicly available nucleic acid and/or peptide sequence cited throughout the disclosure, is/are expressly incorporated herein by reference in their entireties. U.S. Provisional Patent Application No. 62/808,787, filed Feb. 21, 2019, and U.S. Provisional Patent Application No. 62/830,506, filed Apr. 7, 2019, are incorporated by reference in their entireties. The sequence listing filed herewith labeled “WST181PCT_ST25.txt” and the sequences and text therein are incorporated by reference. Embodiments and variations of this invention other than those specifically disclosed above may be devised by others skilled in the art without departing from the true spirit and scope of the invention. The appended claims include such embodiments and equivalent variations.

1. A method of decreasing MDSCs in a subject in need thereof comprising inhibiting, decreasing, deleting, or downregulating FATP2.

2-5. (canceled)

6. The method of claim 1, comprising administering a FATP2 inhibitor to the subject.

7. The method of claim 6, wherein the inhibitor is lipofermata or grassofermata.

8. The method of claim 6, further comprising administering a checkpoint inhibitor to the subject.

9. The method of claim 8, wherein the checkpoint inhibitor is selected from a PD-1 or PD-L1 inhibitor.

10. The method of claim 8, wherein the checkpoint inhibitor binds CTLA4.

11. The method of claim 10, wherein the checkpoint inhibitor is an anti-CTLA4 antibody.

12. The method of claim 6, further comprising administering a CSF-1/1R binding agent or CSF1R inhibitor to the subject.

13. The method of claim 12, wherein the inhibitor is an anti-CSFR1 antibody.

14. The method of claim 6, wherein the subject has cancer.

15. The method of claim 14, wherein the cancer is selected from lymphoma, carcinoma and myeloma.


SEQUENCE LISTING (7 sequences)

SEQ ID NO: 1 - protein, 620 aa, Homo sapiens
MLSAIYTVLAGLLFLPLLVNLCCPYFFQDIGYFLKVAAVGRRVRSYGKRRPARTILRAFLEKARQTPHKPFLLFRDETLTYAQVDRRSNQVARALHDHLGLRQGDCVALLMGNEPAYVWLWLGLVKLGCAMACLNYNIRAKSLLHCFQCCGAKVLLVSPELQAAVEEILPSLKKDDVSIYYVSRTSNTDGIDSFLDKVDEVSTEPIPESWRSEVTFSTPALYIYTSGTTGLPKAAMITHQRIWYGTGLTFVSGLKADDVIYITLPFYHSAALLIGIHGCIVAGATLALRTKFSASQFWDDCRKYNVTVIQYIGELLRYLCNSPQKPNDRDHKVRLALGNGLRGDVWRQFVKRFGDICIYEFYAATEGNIGFMNYARKVGAVGRVNYLQKKIITYDLIKYDVEKDEPVRDENGYCVRVPKGEVGLLVCKITQLTPFNGYAGAKAQTEKKKLRDVFKKGDLYFNSGDLLMVDHENFIYFHDRVGDTFRWKGENVATTEVADTVGLVDFVQEVNVYGVHVPDHEGRIGMASIKMKENHEFDGKKLFQHIADYLPSYARPRFLRIQDTIEITGTFKHRKMTLVEEGFNPAVIKDALYFLDDTAKMYVPMTEDIYNAISAKTLKL

SEQ ID NO: 2 - DNA, 102 bp, Artificial Sequence
  engineered construct
gtccacaataccgtcgatgtgttttagagctagaaatagcaagttaaaataaggctagtccgttatcaacttgaaaaagtggcaccgagtcggtgctttttt

SEQ ID NO: 3 - DNA, 102 bp, Artificial Sequence
  engineered construct
actcctccgttatatgattggttttagagctagaaatagcaagttaaaataaggctagtccgttatcaacttgaaaaagtggcaccgagtcggtgctttttt

SEQ ID NO: 4 - DNA, 160 bp, Artificial Sequence
  engineered construct
tttactttgtttgctttgtgtgttttggtaaatgtcgaactgagtccacaataccgtcgatgtataacttcgtataatgtatgctatacgaagttattggaaagtggctcgcgtaacagaacaaaatctcaaaacaaattaacaggaccccattgctcga

SEQ ID NO: 5 - DNA, 160 bp, Artificial Sequence
  engineered construct
atactgtaatggatggttttaatattctgataaattaaaaatcactcctccgttatatgattgataacttcgtataatgtatgctatacgaagttataggaaacatatagaattttccagcctagctccgtcttcaaagcccacgtttcttatacagtgc

SEQ ID NO: 6 - DNA, 35 bp, Artificial Sequence
  primer sequence
ggtggtcccgggcctatgctgccagtgctctacac

SEQ ID NO: 7 - DNA, 32 bp, Artificial Sequence
  primer sequence
ggtggtgtcgactcagagcttcagagttttat